(19) United States
(12) Patent Application Publication
(10) Pub. No.: US 2025/0340925 A1
(43) Pub. Date: Nov. 6, 2025

(54) NOVEL BEAD LINK (BLINK) METHOD FOR MOLECULAR ARCHIVING OF DNA

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventor: Brian Robert Loomis, New York, NY (US)

(21) Appl. No.: 18/855,516

(22) PCT Filed: Apr. 10, 2023
(86) PCT No.: PCT/US23/18030

§ 371 Date: Oct. 9, 2024

Related U.S. Application Data
(60) Provisional application No. 63/329,775, filed on Apr. 11, 2022.

(51) Int. Cl. C12Q 1/6806; C12Q 1/6811; C12Q 1/6844

(52) U.S. Cl. CPC C12Q 1/6806; C12Q 1/6811; C12Q 1/6844

(57) ABSTRACT

The present disclosure provides methods involving the use of modified magnetic beads for molecular archiving of nucleic acid molecules isolated from a biological sample, such as cell-free DNA (cfDNA). The modified magnetic bead compositions disclosed herein efficiently captured nucleic acid molecules (e.g., DNA) via electrostatic catalysis to generate nucleic acid libraries for iterative molecular analysis.

### CROSS-REFERENCE TO RELATED APPLICATIONS

This application claims the benefit of and priority to U.S. Provisional Patent Application No. 63/329,775, filed Apr. 11, 2022, the contents of which are incorporated herein by reference in its entirety.

### STATEMENT OF GOVERNMENT INTEREST

This invention was made with government support under CA055349, awarded by the National Institutes of Health. The government has certain rights in the invention.

### TECHNICAL FIELD

The present disclosure provides methods involving the use of modified magnetic beads for molecular archiving of nucleic acid molecules isolated from a biological sample, such as cell-free DNA (cfDNA). The modified magnetic bead compositions disclosed herein efficiently captured nucleic acid molecules (e.g., DNA) via electrostatic catalysis to generate nucleic acid libraries for iterative molecular analysis.

### BACKGROUND

The following description of the background of the present technology is provided simply as an aid in understanding the present technology and is not admitted to describe or constitute prior art to the present technology.

When dealing with limited quantities of DNA (e.g. cfDNA), in most cases there is insufficient nucleic acid material to perform all assays of interest because the original nucleic acid material is depleted with each use. In addition, some assays such as methylation analysis assays require the original (not PCR-amplified) DNA molecules, as PCR amplification results in the loss of methylation (and hence loss of signal). Thus, repeatedly sequencing the original DNA molecules from a given sample would overcome many issues related to accurate genetic analysis and mitigate issues with processing small amounts of DNA analyte.

Traditional library preparation methods for iterative molecular analysis exhibit about 30% conjugation efficiency between functionalized detectably labeled agarose beads and 1 μg genomic DNA molecules. See Lau, B. T., & Ji, H. P. (2019). Covalent “Click Chemistry-” Based Attachment of DNA onto Solid Phase Enables Iterative Molecular Analysis. Analytical Chemistry, 91 (3), 1706-1710. Such conjugation efficiencies are grossly unsuitable for iterative molecular analysis involving cell-free DNA (cfDNA) molecules that are present in extremely limited amounts.

Accordingly, there is an urgent need for methods and compositions that efficiently recover/capture original nucleic acid molecules isolated from a subject to generate nucleic acid libraries for iterative molecular analysis.

### Summary of the Present Technology

In one aspect, the present disclosure provides a method including (a) conjugating a heterocyclic amine to a surface of a magnetic bead to obtain a modified magnetic bead having a pH dependent charge state; and (b) conjugating a reagent comprising a first click chemistry reactive group to the surface of the modified magnetic bead, wherein the modified magnetic bead is configured to attach to a nucleic acid molecule comprising a second click chemistry reactive group, wherein the second click chemistry reactive group of the nucleic acid molecule forms a covalent linkage with the first click chemistry reactive group on the surface of the modified magnetic bead. In some embodiments, step (a) and step (b) occur simultaneously or sequentially. In some embodiments, the pH dependent charge state of the modified magnetic bead may be positive at an acidic pH and neutral at a neutral pH.

In some embodiments, the surface of the modified magnetic bead may comprise at least one carboxylate-moiety. In certain embodiments, the reagent comprising the first click chemistry reactive group may be conjugated to the surface of the modified magnetic bead via the at least one carboxylate-moiety. In certain embodiments, the reagent may further comprise a hydrophilic spacer, optionally wherein the hydrophilic spacer comprises one or more of an ethylene glycol moiety (e.g., PEG), a sulfonate moiety, a sulfone moiety, a sulfonyl moiety, a sulfonamide moiety, a phosphate moiety, or a phosphinate moiety.

In some embodiments, the reagent may further comprise one or more functional moieties selected from among dibenzocyclooctyne (DBCO), trans-cyclooctene (TCO), triazole, methyltetrazine, thiol or maleimide. In certain embodiments, the first click chemistry reactive group may be methyltetrazine and the second click chemistry reactive group may be trans-cyclooctene (TCO). Alternatively or additionally, the first click chemistry reactive group may be azide and the second click chemistry reactive group may be dibenzocyclooctyne (DBCO). Alternatively or additionally, the first click chemistry reactive group may be azide and the second click chemistry reactive group may be alkyne. Alternatively or additionally, the first click chemistry reactive group may be maleimide and second click chemistry reactive group may be thiol. Alternatively or additionally, the first click chemistry reactive group may be trans-cyclooctene (TCO) and the second click chemistry reactive group may be methyltetrazine. Alternatively or additionally, the first click chemistry reactive group may be dibenzocyclooctyne (DBCO) and the second click chemistry reactive group may be azide. Alternatively or additionally, the first click chemistry reactive group may be alkyne and the second click chemistry reactive group may be azide. Alternatively or additionally, the first click chemistry reactive group may be thiol and second click chemistry reactive group may be maleimide.

In some embodiments, the heterocyclic amine may be 2-(2-aminoethyl)pyridine or 2-(2-aminoethyl)imidazole. In one aspect, the present disclosure provides a modified magnetic bead produced by any and all embodiments of the method disclosed herein.

In one aspect, the present disclosure provides a method for archiving nucleic acid molecules isolated from a biological sample comprising (a) isolating a nucleic acid molecule from a biological sample; (b) ligating an adapter to at least one strand of the isolated nucleic acid molecule to form an adapter-tagged nucleic acid molecule, wherein the adapter comprises a click chemistry ligand; and (c) coupling the adapter-tagged nucleic acid molecule to any and all embodiments of the modified magnetic bead described herein to form an adapter-tagged nucleic acid-bead complex, wherein the click chemistry ligand of the adapter forms a covalent linkage with the first click chemistry reactive group on the surface of the modified magnetic bead. The isolated nucleic acid molecule may be double-stranded DNA, single stranded DNA, double-stranded RNA or single stranded RNA. In some embodiments, the double-stranded DNA is genomic DNA, cell-free DNA, or ctDNA. Additionally or alternatively, the isolated nucleic acid molecule may be obtained from a nucleic acid library. In certain embodiments, the method further comprises generating copies of the isolated nucleic acid molecule (e.g., via PCR or isothermal strand displacement) prior to performing step (b).

In some embodiments, the method further comprises contacting the adapter-tagged nucleic acid-bead complex with a blocking agent. Non-limiting examples of suitable blocking agents include, but are not limited to surfactants (e.g., Triton X-100, Tween® 20), polymers (e.g., polyethylene glycol (PEG), polyvinyl alcohol (PVA), polyvinylpyrrolidone (PVP), Ficoll), bovine serum albumin (BSA), coldwater fish gelatine, tryptone casein peptone, casein, milk, serum, and nucleic acid blocking agents (e.g., salmon sperm DNA, calf thymus DNA, yeast tRNA, homopolymer DNA, herring sperm DNA, total human DNA, COT1 DNA). Additionally or alternatively, in some embodiments, the adapter-tagged nucleic acid-bead complex is contacted with the blocking agent for about 30-90 minutes at a temperature of about 20° C.-65° C. In certain embodiments, the adapter-tagged nucleic acid-bead complex is contacted with the blocking agent for about 30-35 minutes, about 35-40 minutes, about 40-45 minutes, about 45-50 minutes, about 50-55 minutes, about 55-60 minutes, about 60-65 minutes, about 65-70 minutes, about 70-75 minutes, about 75-80 minutes, about 80-85 minutes, or about 85-90 minutes at a temperature of about 20° C.-25° C., about 25° C.-30° C., about 30° C.-35° C., about 35° C.-40° C., about 40° C.-45° C., about 45° C.-50° C., about 55° C.-60° C., or about 60° C.-65° C.

Additionally or alternatively, in some embodiments, the method further comprises directly amplifying the adapter-tagged nucleic acid molecule that is coupled to the modified magnetic bead to obtain amplicons. In other embodiments, the method further comprises generating at least one bead-linked copy strand from the adapter-tagged nucleic acid molecule that is coupled to the modified magnetic bead and amplifying the at least one bead-linked copy strand to obtain amplicons. In any of the preceding embodiments of the methods disclosed herein, amplicons may be generated using one or more of the following PCR conditions: an annealing step of about 1, 2, 3, 4, 5, 6, 7, 8, 9 or 10 minutes per cycle, an extension step of about 1, 2, 3, 4 or 5 minutes per cycle, a final extension step of about 5, 6, 7, 8, 9 or 10 minutes.

In any of the preceding embodiments, the method further comprises (a) sequencing the amplicons; (b) detecting at least one genetic alteration in the amplicons, optionally wherein the at least one genetic alteration is selected from the group consisting of a single nucleotide variant (SNV), a copy number variant (CNV), an insertion, a deletion, a duplication, an inversion, a translocation and a gene fusion; and/or (c) enriching the amplicons with a selector comprising a set of oligonucleotides that selectively hybridize to genomic regions of one or more target genes, optionally wherein the one or more target genes correspond to cancer-related genes.

In certain embodiments, the amplicons are sequenced using high throughput, massively parallel sequencing (i.e., next generation sequencing). Methods for performing high throughput, massively parallel sequencing are known in the art. In some embodiments of the method, the high throughput massive parallel sequencing is performed using 454™ GS FLX™ pyrosequencing, reversible dye-terminator sequencing, SOLID sequencing, Ion semiconductor sequencing, Helioscope single molecule sequencing, sequencing by synthesis, sequencing by ligation, or SMRT™ sequencing. In some embodiments, high throughput massively parallel sequencing may be performed using a read depth approach.

In any of the above embodiments, the method further comprises detecting DNA methylation in the adapter-tagged nucleic acid molecule that is coupled to the modified magnetic bead via sodium bisulfite conversion and sequencing, Differential methylation hybridization (DMH), or affinity capture of methylated DNA.

Additionally or alternatively, in some embodiments of the methods disclosed herein, the adapter further comprises a PCR primer binding site, a sequencing primer binding site, or any combination thereof. Additionally or alternatively, in certain embodiments of the methods disclosed herein, the adapter further comprises a sample-specific barcode sequence, wherein the sample-specific barcode sequence comprises 2-20 nucleotides, and/or a detectable label.

In any and all embodiments of the methods disclosed herein, the biological sample comprises no more than 5 ng of cell-free DNA or at least 6-30 ng of cell-free DNA. Additionally or alternatively, in some embodiments of the methods disclosed herein, the biological sample is whole blood, serum, plasma, synovial fluid, lymphatic fluid, ascites fluid, interstitial fluid or a biopsied tissue sample. In certain embodiments, the biological sample is obtained from a patient. Additionally or alternatively, in some embodiments, the patient is diagnosed with ovarian cancer, breast cancer, colon cancer, lung cancer, prostate cancer, gastric cancer, pancreatic cancer, cervical cancer, liver cancer, bladder cancer, cancer of the urinary tract, thyroid cancer, renal cancer, carcinoma, melanoma, head and neck cancer, or brain cancer.

### BRIEF DESCRIPTION OF THE DRAWINGS

FIG. 1: Conjugation of heterocyclic amines to carboxylate-modified magnetic particles produces a magnetic substrate where the surface charge can be modulated by buffer pH. Buffer pH values lower than the pKa of the heterocycle results in a positively charged surface, whereas pH values above the pKa results in a non-protonated and therefore neutral surface. At lower pH values, the positive surface of the magnetic particle can electrostatically attract the polyanionic backbone of DNA and RNA.

FIG. 2: In addition to adding heterocyclic amines to the bead surface, other chemical reactive groups (e.g. click chemistry ligands) can be added to bead surface using standard conjugation chemistry to create a bifunctional modified magnetic bead of the present technology.

FIG. 3: Creation of a bifunctional modified magnetic bead of the present technology. The modified magnetic beads of the present technology permit rapid nucleic acid bead immobilization to the bead surface (<1 min). The nucleic acid capture is highly efficient and nearly quantitative, even for small quantities of nucleic acids.

FIG. 4: Exemplary chemical reaction groups that can be used in the nucleic acid-bead linking methods disclosed herein. Click chemistry groups allow for rapid covalent conjugation of the DNA substrate to the bead surface.

FIG. 5: Rate-Enhanced Click Reactions via Electrostatic Catalysis. Under neutral conditions, there is no attractive force between the labeled DNA and the magnetic particle.

FIG. 6: A slight drop in pH charges the surface of the beads, creating an attractive force between the bead and the labeled DNA. Once the DNA is attractive to/electrostatically adhered to the bead, the click chemistry group on the DNA can react with the corresponding reactive group on the bead. In essence, the electrostatic attraction ‘concentrates’ the click substrates, resulting in a dramatic increase in the reaction rate of the reagents. The overall process creates an incredibly fast conjugation reaction.

FIGS. 7-8: One embodiment of a bifunctional modified magnetic bead disclosed herein involves conjugation of 2-amino pyridine along with amino-PEG4-azide to 1 μm magnetic particles. After the first conjugation, the azide functionality is converted into a TCO group by reaction with DBCO-PEG4-TCO, and the final bead is washes to remove all unbound reagents.

FIGS. 9-10: Efficient Synthesis of Lab-Made mTz Adapters. To install a click group on the DNA used for the nucleic acid-bead linking methods of the present technology, azide-labeled oligos are incubated overnight with bifunctional click reagents (e.g. mTz-DBCO) to create the proper click reagent. Successful modification of the oligo results in an electrophoretic mobility shift. Installation of a click group into an oligo used for NGS library preparation allows for the incorporation of the click group into the native DNA molecules. Installation of the click group on oligos used for PCR allow for labeling of PCR products for testing purposes. SPAAC Ligand=Strain-promoted azide-alkyne cycloaddition, iEDDA=Inverse electron demand Diels-Alder reactions.

FIG. 11: An amplicon was made with a methyltetrazine (mTz) click group on one strand. This mTz labeled amplicon and an un-labeled amplicon that does not have the click group (Standard or Std) underwent electrostatic catalysis. These amplicons were then washed five times with TE/SDS/NaCl. The supernatant(S) and washes were saved and PCR was performed to determine the amount of material on the bead (mTz BLINK and Std BLINK), and the amount of material left in the supernatant and washes. PCR controls were run to make sure the reagents were not faulty. Most of the material for the mTz BLINK reaction ends up on the bead, whereas most of the material of the unlabeled material ends up in the washes. This shows that the nucleic acid bead linking methods of the present technology is highly specific for the click chemistry reaction.

FIG. 12: In order for the DNA to be captured onto the bead, electrostatic catalysis is required. An amplicon was made with a methyltetrazine (mTz) click group on the both strands. One set did not have the binding buffer added to it (No Electrostatic Catalysis), whereas the other did (Electrostatic Catalysis). The reactions were allowed to react overnight. These amplicons were then washed three times with 0.5 M NaOH/Tween. The supernatant(S) and washes (W1, W2, and W3) were saved and PCR was performed to see the amount of material on the bead (B), and the amount of material left in the supernatant and washes. A PCR control was run to make sure the reagents were not faulty. Without electrostatic catalysis, the material will not bind to the bead, as can be seen by S in No Electrostatic Catalysis. The opposite is true for the sample with Electrostatic Catalysis as most of the material is seen on bead (B-Electrostatic Catalysis).

FIG. 13: Based on previous technology, higher duplex family counts correlates with higher library complexity and recovery of both strands of the original DNA molecule. This can be represented as extrapolated duplex family counts over total reads or as unique duplex families. Data can be visualized as the duplex family extrapolation, or as a single data point on the extrapolation curve at a set read depth (e.g. 5×10⁸).

FIG. 14: In order to ensure that the click group would not interfere with library preparation, NGS libraries were made using the Kapa Hyper Kit, the standard IDT Prism kit, and the IDT prism kit with a mTz modified adapter, and sequenced. The click group does not affect the performance of the library preparation kit as can be seen by the similar duplex family count as the standard IDT Prism kit. Installation of the click group does not negatively affect library prep. The data here is extrapolated library complexity at a total read depth of 5e10\hat{ }8

FIG. 15: The data recovered from the nucleic acid library archived on the modified magnetic beads (mTz BLINK) is lower in complexity relative to the libraries made using standard library preparation kits. In these experiments, the library was amplified directly from the modified magnetic beads. Since archiving the DNA onto the surface appears efficient, this is most likely due to the inefficiency of reading the material off the beads.

FIG. 16: Generation of a bead-linked copy strand improves reading of the archived material from the bead. During the modified library preparation, Bst 2.0 was used to separate the strands and a primer was added to create bead-linked copy strands. NaOH washes were then performed to strip the bead-linked copy strand before performing PCR. The data here is represented as an extrapolation.

FIGS. 17-18: Five individual sequencing libraries were iteratively generated from the same bead. None of the libraries by themselves show complexity (duplex family content) that equals the control ‘Prism’ preparation (NGS library made with standard IDT prism kit). Information content in each library is additive (to a point). However, combining these libraries together shows that each library contains complementary DNA information and produces better duplex yield. Therefore multiple sampling will improve the complexity of the final library and may bring the value close to that of a control NGS library made with standard IDT prism kit.

FIG. 19: shows a schematic of an exemplary workflow for iterative molecular analysis. Adapted from Lau & Ji, Analytical Chemistry, 91 (3), 1706-1710 (2019).

FIG. 20: Strand separation prior to linking adapter-tagged nucleic acids to the modified magnetic beads of the present technology improves duplex count. Compared to a control library, a bead linked (BLINK)-archived nucleic acid library shows less duplex molecule content (first and second columns). When the original DNA strands are separated by either isothermal strand displacement/polymerization (for example, using the Bst2.0 enzyme) or by limited rounds of PCR (e.g. pre-amplification), the resulting archived libraries show higher duplex content (columns 3 and 4).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

One of the key challenges for NGS analysis involves the scarce quantities of nucleic acid material from specific samples; examples include biopsies of disease tissue or circulating DNA isolated from blood plasma. Limited amounts of tissue samples frequently yield enough DNA or RNA for only a single assay, thus limiting the breadth of analyses that can be performed. Iterative, replicated analysis of original DNA molecules from a given sample would overcome many issues related to accurate genetic analysis and mitigate issues with processing small amounts of DNA analyte. Traditional library preparation methods for iterative molecular analysis exhibit about 30% conjugation efficiency between functionalized detectably labeled agarose beads and 1 μg genomic DNA molecules. See Lau, B. T., & Ji, H. P. (2019). Covalent “Click Chemistry-” Based Attachment of DNA onto Solid Phase Enables Iterative Molecular Analysis. Analytical Chemistry, 91 (3), 1706-1710; FIG. 19. Such conjugation efficiencies are grossly unsuitable for iterative molecular analysis involving cell-free DNA (cfDNA) molecules that are present in extremely limited amounts.

The methods disclosed herein are useful for recovering and archiving original nucleic acid (e.g., DNA) molecules isolated from a biological sample onto the surface of magnetic beads for the purpose of iterative molecular analysis. The modified magnetic bead compositions of the present technology efficiently captured nucleic acid molecules (e.g., >99% conjugation efficiency) via electrostatic catalysis to generate nucleic acid libraries for iterative molecular analysis. Thus, with the original nucleic acid (e.g., DNA) molecules permanently attached to the surface, one may perform many sequential analyses from the same input nucleic acid material, since the original nucleic acid molecules are always retained, and the analysis is performed on a PCR-amplified copy of the original nucleic acid molecule. Moreover, the methods of the present technology permit multiple analysis, followed by methylation analysis, since the methylation patterns on the original nucleic acid molecules are retained on the surface of the beads.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)—CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, and —C≡CCH₂CH(CH₂CH₃)₂, among others.

Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3—, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3—, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkyloyl” and “alkyloyloxy” as used herein can refer, respectively, to —C(O)-alkyl groups and —O—C(O)-alkyl groups. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O) H). In some embodiments, the amide is —NR⁷¹C(O)-(C₁₋₅ alkyl) and the group is termed “carbonylamino,” and in others the amide is —NHC(O)-alkyl and the group is termed “alkanoylamino.”

The term “nitrile” or “cyano” as used herein refers to the—CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, “sulfonyls” include —SO₂OR⁸³, and “sulfonates” include —SO₃″. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰° C. (NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴° C. (R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂-.

The term “imide” refers to —C(O)NR⁹⁸° C. (O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR 100 (NR¹⁰¹) and —N(CR 100R 101) groups, wherein R 100 and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl) 3 group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the 3′ or 5′ end of a nucleic acid sequence in order to facilitate attachment to another molecule. The adapter can be single-stranded or double-stranded. An adapter may incorporate a short (e.g., less than 55 base pairs) sequence useful for PCR amplification or sequencing. The adapter can comprise known sequences, degenerate sequences (a sequence not having a precise definition), or both. A double-stranded adapter may comprise two hybridizable strands. Alternatively, a double-stranded adapter can comprise a hybridizable portion and a non-hybridizable portion. The non-hybridizable portion of a double-stranded adapter comprises two single-stranded regions that are not hybridizable to each other. Within the non hybridizable portion, the strand containing an unhybridized 5′-end is referred to as the 5′-strand and the strand containing an unhybridized 3′-end is referred to as the 3′-strand. In some embodiments, the double-stranded adapter has a hybridizable portion at one end of the adapter and a non-hybridizable portion at the opposite end of the adapter. In some embodiments, the non-hybridizable portion of the double-stranded adapter may be open (Y-shaped adapter). In some embodiments, the adapter may be a U-shaped adapter. Additionally or alternatively, in some embodiments, the adapters further comprise a click chemistry reactive group (e.g., an azide, methyltetrazine (mTz) etc.). Additionally or alternatively, in some embodiments, the adapters further comprise a hydrophilic spacer, such as PEG spacer.

As used herein, the terms “amplify” or “amplification” with respect to nucleic acid sequences, refer to methods that increase the representation of a population of nucleic acid sequences in a sample. Nucleic acid amplification methods, such as PCR, isothermal methods, rolling circle methods, etc., are well known to the skilled artisan. Copies of a particular nucleic acid sequence generated in vitro in an amplification reaction are called “amplicons” or “amplification products”.

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

“Bait”, as used herein, is a type of hybrid capture reagent that retrieves target nucleic acid sequences for sequencing. A bait can be a nucleic acid molecule, e.g., a DNA or RNA molecule, which can hybridize to (e.g., be complementary to), and thereby allow capture of a target nucleic acid. In one embodiment, a bait is an RNA molecule (e.g., a naturally-occurring or modified RNA molecule); a DNA molecule (e.g., a naturally-occurring or modified DNA molecule), or a combination thereof. In other embodiments, a bait includes a binding entity, e.g., an affinity tag, that allows capture and separation, e.g., by binding to a binding entity, of a hybrid formed by a bait and a nucleic acid hybridized to the bait. In one embodiment, a bait is suitable for solution phase hybridization.

The term “barcode” refers to a sequence of nucleotides within a polynucleotide that is used to identify a nucleic acid molecule. For example, a barcode can be used to identify molecules when the molecules from several groups are combined for processing or sequencing in a multiplexed fashion. A barcode can be located at a certain position within a polynucleotide (e.g., at the 3′-end, 5′-end, or middle of the polynucleotide) and can comprise sequences of any length (e.g., 1-100 or more nucleotides). Additionally, a barcode can comprise one or more pre-defined sequences. The term “pre-defined” means that sequence of a barcode is predetermined or known prior to identifying or without the need to identify the entire sequence of the nucleic acid comprising the barcode. In some cases, pre-defined barcodes can be attached to nucleic acids for sorting the nucleic acids into groups. In some embodiments, a barcode can comprise artificial sequences, e.g., designed or engineered sequences that are not present in the unaltered (wild-type) genome of a subject. In other embodiments, a barcode can comprise an endogenous sequence, e.g., sequences that are present in the unaltered (wildtype) genome of a subject. In certain embodiments, a barcode can be an endogenous barcode. An endogenous barcode can be a sequence of a genomic nucleic acid, where the sequence is used as a barcode or identifier for the genomic nucleic acid. One or more sequences of the genomic DNA fragment can be an endogenous barcode. Different types of barcodes can be used in combination. For example, an endogenous genomic nucleic acid fragment can be attached to an artificial sequence, which can be used as a unique identifier of the genomic nucleic acid fragment. A “sample-specific barcode” or “patient barcode” refers to a polynucleotide sequence that is used to identify the origin or source of a nucleic acid molecule. For example, a sequence of “AAAA” can be attached to identify nucleic acids isolated from Patient A.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer cells” includes precancerous (e.g., benign), malignant, pre-metastatic, metastatic, and non-metastatic cells. Cancers of virtually every tissue are known to those of skill in the art, including solid tumors such as carcinomas, sarcomas, glioblastomas, melanomas, lymphomas, myelomas, etc., and circulating cancers such as leukemias. Examples of cancer include, but are not limited to, ovarian cancer, breast cancer, colon cancer, lung cancer, prostate cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer. The phrase “cancer burden” or “tumor burden” refers to the quantity of cancer cells or tumor volume in a subject. Reducing cancer burden accordingly may refer to reducing the number of cancer cells, or the tumor volume in a subject. The term “cancer cell” refers to a cell that exhibits cancer-like properties, e.g., uncontrollable reproduction, resistance to anti-growth signals, ability to metastasize, and loss of ability to undergo programmed cell death (e.g., apoptosis) or a cell that is derived from a cancer cell, e.g., clone of a cancer cell.

The term “cell-free DNA (cfDNA)” refers to DNA in a sample that when collected, was not contained within a cell. cfDNAs can comprise both normal cell and cancer cell-derived DNA. cfDNA is commonly obtained from blood or plasma (“circulation”). cfDNAs may be released into the circulation through secretion or cell death processes, e.g., cellular necrosis or apoptosis. A fraction of cfDNA may include ctDNA.

The term “circulating tumor DNA (ctDNA)” refers to the fraction of cell-free DNA (cfDNA) in a sample that originates from a tumor.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state).

As used herein, a “bead-linked copy strand” refers to a copy of a strand of an adapter-tagged nucleic acid molecule coupled to a magnetic bead. In some embodiments, the bead-linked copy strand may further comprise a detectable label.

“Detecting” as used herein refers to determining the presence of a mutation in a nucleic acid of interest in a sample. Detection does not require the method to provide 100% sensitivity.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “gene region” can refer to a range of sequences within a gene or surrounding a gene, e.g., an intron, an exon, a promoter, a 3′ untranslated region etc.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

The term “hybridizable” means that two polynucleotide strands of a nucleic acid are complementary at one or more nucleotide positions, e.g., the nitrogenous bases of the two polynucleotide strands can form two or more Crick-Watson hydrogen bonds. For example, if a polynucleotide comprises 5′ ATGC 3′, it is hybridizable to the sequence 5′ GCAT 3′. Under some experimental conditions, if a polynucleotide comprises 5′ GGGG 3′, it can also be hybridizable to the sequences 5′CCAC 3′ and 5′ CCCA 3′, which are not perfectly complementary.

The term “non-hybridizable” means that two polynucleotide strands of a nucleic acid are non-complementary, e.g., nitrogenous bases of the two separate polynucleotide strands do not form two or more Crick-Watson hydrogen bonds under stringent hybridization conditions.

As used herein, the terms “individual”, “patient”, or “subject” can be an individual organism, a vertebrate, a mammal, or a human. In some embodiments, the individual, patient or subject is a human.

As used herein, the term “library” refers to a collection of nucleic acid sequences, e.g., a collection of nucleic acids derived from whole genomic, subgenomic fragments, cDNA, cDNA fragments, cfDNA, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, for sequencing, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject. In one embodiment, the subject has, or is at risk of having, a cancer or tumor.

A “library nucleic acid sequence” refers to a nucleic acid molecule, e.g., DNA, RNA, or a combination thereof, that is a member of a library. In some embodiments, a library nucleic acid sequence is a DNA molecule, e.g., genomic DNA, cfDNA, or cDNA. In some embodiments, a library nucleic acid sequence is fragmented, e.g., sheared or enzymatically prepared, genomic DNA. In certain embodiments, the library nucleic acid sequences comprise a sequence from a subject and a sequence not derived from the subject, e.g., adapter sequence, a primer sequence, or other sequences that allow for identification, e.g., “barcode” sequences.

The term “ligating” refers to connecting two molecules by chemical bonds to generate a new molecule. For example, ligating an adapter polynucleotide to another polynucleotide can refer to forming chemical bonds between the adapter and the polynucleotide (e.g., using a ligase or any other method) to generate a single new molecule comprising the adapter and the polynucleotide.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. Oligonucleotides of the method which function as primers or probes are generally at least about 10-15 nucleotides long and more preferably at least about 15 to 55 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

As used herein, a “selector” refers to a plurality of oligonucleotides or probes that hybridize with one or more genomic regions. In some embodiments, the one or more genomic regions may be associated with diseases, e.g., cancers.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

The term “spacer” or spacer-moiety as used herein is a chemical moiety that spaces (i.e., provides distance between) and covalently links together two (or more) parts of a connecting group. A spacer may be hydrophilic or hydrophobic. A hydrophilic spacer may comprise, for example, one or more units, or combinations thereof, selected from ethylene glycol, a carboxylic acid moiety, a sulfonate moiety, a sulfone moiety, a sulfonyl moiety, a sulfonamide moiety, a phosphate moiety, a phosphinate moiety, or an amino group.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

Those of skill in the art will appreciate that compounds of the present technology may exhibit the phenomena of tautomerism, conformational isomerism, geometric isomerism, and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

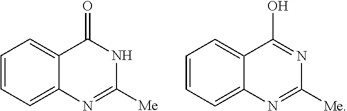

As another example, guanidines may exhibit the following isomeric forms in protic organic solution, also referred to as tautomers of each other:

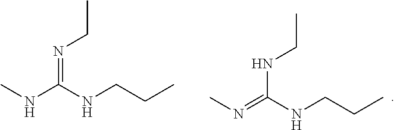

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

### Samples

Samples may be collected from subjects repeatedly over a period of time (e.g., once a day, once a week, once a month, biannually or annually). Obtaining numerous samples from a subject over a period of time can be used to verify results from earlier detections or to identify an alteration as a result of, for example, drug treatment.

The sample may comprise nucleic acids including tumor nucleic acids. The nucleic acids may be genomic nucleic acids. The nucleic acids may also be circulating nucleic acids, e.g., cell-free nucleic acids. For example, the circulating nucleic acids may be from a tumor, e.g., ctDNA. Sample nucleic acids useful for the methods of the present technology may comprise cfDNA, e.g., DNA in a sample that is not contained within a cell. Such DNA may be fragmented, e.g., may be on average about 170 nucleotides in length, which may coincide with the length of DNA wrapped around a single nucleosome.

cfDNA may be a heterogeneous mixture of DNA from normal and tumor cells, and an initial sample of cfDNA may not be enriched for cancer cell DNA and recurrently mutated regions of a cancer cell genome. One of skill in the art will understand that non-mutated germline sequences may not be distinguished between a tumor source and a normal cell source, but sequences containing somatic mutations have a probability of being derived from tumor DNA. In some embodiments, a sample may comprise control germline DNAs. A sample may also comprise known tumor DNA. Further, a sample may comprise cfDNA obtained from an individual suspected of having ctDNA in the sample. Additionally, a sample may comprise cfDNA obtained from an individual not suspected of having ctDNA in the sample, for example, as part of routine testing.

The methods disclosed herein may comprise obtaining one or more samples, e.g., nucleic acid samples, from a subject. The one or more sample nucleic acids may be tumor nucleic acids. For example, nucleic acids may be extracted from tumor biopsies. Tumor nucleic acids may also be released into the blood stream from tumor cells, e.g., as a result of immunological responses to the tumor. The tumor nucleic acid that is released into the blood can be ctDNA.

The one or more sample nucleic acids may be genomic nucleic acids. It should be understood that the step of obtaining tumor nucleic acids and genomic nucleic acids from a subject with a specific cancer may occur simultaneously. For example, venipuncture to collect blood, plasma, or serum, may simultaneously collect both genomic and tumor nucleic acids. Obtaining tumor nucleic acids and genomic nucleic acids from a subject with a specific cancer may also occur at separate occasions. For example, it may be possible to obtain a single tissue sample from a patient, for example, a biopsy sample, which includes both tumor nucleic acids and genomic nucleic acids. It is also possible to obtain the tumor nucleic acids and genomic nucleic acids from the subject in separate samples, in separate tissues, or at separate times.

Obtaining tumor nucleic acids and genomic nucleic acids from a subject with a specific cancer may also include the process of extracting a biological fluid or tissue sample from the subject with the specific cancer. Obtaining the nucleic acids may include procedures to improve the yield or recovery of the nucleic acids, such as separating the nucleic acids from other cellular components and contaminants that may be present in the biological fluid or tissue sample, e.g., by phenol chloroform extraction, precipitation by organic solvents, or DNA-binding spin columns.

Sometimes, the nucleic acids are mixed or impure. In some embodiments, two or more samples may be isolated from two or more subjects. Patient barcode sequences may be employed to identify a sample from which the nucleic acid originated and to sort the nucleic acids into different groups. In some embodiments, nucleic acids from a first sample may be associated with a first patient barcode, whereas nucleic acids from a second sample may be associated with a second patient barcode.

In other embodiments, the two or more samples may be from the same subject. In certain embodiments, the two or more samples may be from different tissues of the same subjects. For example, one sample may be from a tumor (e.g., a solid tumor) and another sample may be from the blood of the same subject. The samples may be obtained at the same time or at two or more time points.

### Preparation of Nucleic Acid Libraries

Fragmentation. In some embodiments, the isolated nucleic acids from a biological sample are fragmented, e.g., sheared or enzymatically prepared, prior to nucleic acid library preparation.

Adapter Ligation. In some embodiments, an adapter is ligated to the 3′ and/or 5′ end of at least one strand of the isolated nucleic acids from a biological sample. In any and all embodiments of the nucleic acid library preparation methods disclosed herein, the adapter comprises a click chemistry ligand. Examples of suitable click chemistry ligands include those described herein as well as other click chemistry reactive groups known in the art. The adapter can be single-stranded or double-stranded. In some embodiments, the adapter may be a U-shaped adapter or a Y-shaped adapter (e.g., a full-length Y-shaped adapter, a stubby-Y adapter). Additionally or alternatively, in certain embodiments, the adapters of the present technology further comprise a hydrophilic spacer, e.g., a PEG spacer. In certain embodiments, the adapter may comprise 1, 2, 3, 4, 5, 6, 7, 8, 9, 10, 11, 12, 13, 14, 15, 16, 17, 18, 19, 20, 21, 22, 23 or 24 PEG units.

Amplification. Additionally or alternatively, in some embodiments, the isolated nucleic acids from a biological sample are amplified prior to nucleic acid library preparation. In some embodiments, the isolated nucleic acids being amplified can be DNA, including genomic DNA, cDNA (complementary DNA), cell-free DNAs (cfDNA) and circulating tumor DNAs (ctDNA). The nucleic acids being amplified can also be RNA. As used herein, one amplification reaction may consist of many rounds of DNA synthesis.

The library preparation methods disclosed herein may comprise amplification of the template nucleic acids comprising sample nucleic acids attached to adapters. Any known techniques for nucleic acid (e.g., DNA and RNA) amplification can be used with the assays described herein. Some amplification techniques are the polymerase chain reaction (PCR) methodologies which can include, but are not limited to, solution PCR and in situ PCR. Alternatively, amplification may comprise isothermal strand displacement or non-exponential amplification, such as linear amplification.

Amplification of the template nucleic acid may comprise the use of one or more polymerases. The polymerase may be a DNA polymerase or an RNA polymerase. In some embodiments, the polymerase may be a high fidelity polymerase, KAPA HiFi DNA polymerase. The polymerase may also be Phusion DNA polymerase.

In some embodiments, a single primer or one or both primers of a primer pair comprise a specific sequencing adapter. This sequencing adapter is a short oligonucleotide of known sequence that can provide a priming site for both amplification and sequencing of the adjoining, target nucleic acid. As such, sequencing adapters allow binding of a fragment to a flow cell for next generation sequencing.

In some embodiments, all forward amplicons (i.e., amplicons extended from forward primers that hybridized with antisense strands of a target nucleic acid) contain the same sequencing adapter. In some embodiments when double stranded sequencing is performed, all forward amplicons contain the same sequencing adapter and all reverse amplicons (i.e., amplicons extended from reverse primers that hybridized with sense strands of a target segment) contain a sequencing adapter that is different from the sequencing adapter of the forward amplicons.

In some embodiments, the sequencing adapters are P5 and/or P7 adapter sequences that are recommended for Illumina sequencers (MiSeq and HiSeq). See, e.g., Williams-Carrier et al., Plant J., 63 (1): 167-77 (2010). In some embodiments, the sequencing adapters are P1, A, or Ion Xpress™ barcode adapter sequences that are recommended for Life Technologies sequencers. Other sequencing adapters are known in the art.

Additionally or alternatively, in some embodiments of the above methods, amplicons from more than one sample are sequenced. In some embodiments, all samples are sequenced simultaneously in parallel. In some embodiments of the above methods, amplicons from at least 1, 5, 10, 20, 30, or up to 35, 40, 45, 48 or 50 different samples are amplified and sequenced using the methods described herein.

Additionally or alternatively, in some embodiments of the method, amplicons derived from a single sample may further comprise an identical index sequence that indicates the source from which the amplicon is generated, the index sequence for each sample being different from the index sequences from all other samples. As such, the use of index sequences permits multiple samples to be pooled per sequencing run and the sample source subsequently ascertained based on the index sequence. In some embodiments, the Access Array™ System (Fluidigm Corp., San Francisco, CA) or the Apollo 324 System (Wafergen Biosystems, Fremont, CA) is used to generate a barcoded (indexed) amplicon library by simultaneously amplifying the nucleic acids from the samples in one set up.

In some embodiments, indexed amplicons are generated using primers (for example, forward primers and/or reverse primers) containing the index sequence. Such indexed primers may be included during library preparation as a “barcoding” tool to identify specific amplicons as originating from a particular sample source. When sequencing adapter-ligated and/or indexed primers are employed, the sequencing adapter and/or index sequence gets incorporated into the amplicon during amplification. Therefore, the resulting amplicons are sequencing-competent and do not require the traditional library preparation protocol. Moreover, the presence of the index tag permits the differentiation of sequences from multiple sample sources. In some embodiments, the amplicon library is generated using a multiplexed PCR approach.

Indexed amplicons from more than one sample source are quantified individually and then pooled prior to high throughput sequencing. As such, the use of index sequences permits multiple samples (i.e., samples from more than one sample source) to be pooled per sequencing run and the sample source subsequently ascertained based on the index sequence. When indexed primer sets are used, this capability can be exploited for comparative studies. In some embodiments, amplicon libraries from up to 48 separate sources are pooled prior to sequencing.

In other embodiments, the employed primers do not contain adapter sequences and the amplicons produced are subsequently (i.e. after amplification) ligated to an oligonucleotide sequencing adapter on one or both ends of the amplicons. In some embodiments, all forward amplicons (i.e., amplicons extended from forward primers that hybridized with antisense strands of a target nucleic acid) contain the same adapter sequence. In some embodiments when double stranded sequencing is performed, all forward amplicons contain the same adapter sequence and all reverse amplicons (i.e., amplicons extended from reverse primers that hybridized with sense strands of a target segment) contain an adapter sequence that is different from the adapter sequence of the forward amplicons.

In some embodiments, the amplicons may be amplified with non-adapter-ligated and/or non-indexed primers and a sequencing adapter and/or an index sequence may be subsequently ligated to one or both ends of each of the resulting amplicons. In some embodiments, the amplicon library is generated using a multiplexed PCR approach.

Indexed amplicons from more than one sample source are quantified individually and then pooled prior to high throughput sequencing. As such, the use of index sequences permits multiple samples (i.e., samples from more than one sample source) to be pooled per sequencing run and the sample source subsequently ascertained based on the index sequence.

End-repair. The methods disclosed herein may comprise performing an end repair reaction on isolated nucleic acids (e.g., cfDNA) to produce a plurality of end repaired nucleic acids. For example, the end repair reaction may be conducted prior to attaching the adapters to the isolated nucleic acids from a biological source.

In some embodiments, the end repair reaction may be conducted prior to amplification of the adapter-tagged nucleic acids. In other embodiments, the end repair reaction may be conducted after amplification of the adapter-tagged nucleic acids.

In some embodiments, the end repair reaction may be conducted prior to fragmenting the isolated nucleic acids from a biological source. In other embodiments, the end repair reaction may be conducted after fragmenting the isolated nucleic acids from a biological source.

The end repair reaction may also be performed by using one or more end repair enzymes. In some embodiments, enzymes for repairing DNA can comprise polymerase and exonuclease. For example, polymerase can fill in the missing bases for a DNA strand from 5′ to 3′ direction. The resulting double-stranded DNA can be the same length as the original longest DNA strand. Exonuclease can remove the 3′ overhangs. The resulting double-stranded DNA can be the same length as the original shortest DNA strand.

A-tailing. The methods disclosed herein may comprise performing an A-tailing reaction on the isolated nucleic acids (e.g., cfDNA) from a biological source to produce a plurality of A-tailed nucleic acids. For example, the A-tailing reaction may be conducted prior to attaching the adapters of the present technology to the isolated nucleic acids.

Further, the A-tailing reaction may be conducted prior to amplification of the adapter-tagged nucleic acids. In other embodiments, the A-tailing reaction may be conducted after amplification of the adapter-tagged nucleic acids.

In some embodiments, the A-tailing reaction may be conducted prior to fragmenting the isolated nucleic acids (e.g., cfDNA) from a biological source. In some cases, the A-tailing reaction may be conducted after fragmenting the isolated nucleic acids (e.g., cfDNA) from a biological source.

In other embodiments, the A-tailing reaction may be conducted prior to end repair of the isolated nucleic acids (e.g., cfDNA) from a biological source. In some embodiments, the A-tailing reaction may be conducted after end repair of the isolated nucleic acids (e.g., cfDNA) from a biological source.

The A-tailing reaction may also be performed by using one or more A-tailing enzymes. For example, an A residue can be added by incubating a DNA fragment with dATP and a non-proofreading DNA polymerase, which will add a single 3′ “A” residue.

### NGS Platforms

Genotyping, detection, identification or quantitation of the ctDNA can utilize sequencing. Sequencing can be accomplished using high-throughput massively parallel sequencing. Sequencing can be performed using nucleic acids described herein such as genomic DNA, cfDNA, cDNA derived from RNA transcripts or RNA as a template. For example, sequence information of the cell-free DNA sample may be obtained by massively parallel sequencing. In some embodiments, massively parallel sequencing may be performed on a subset of a genome, e.g., from a subset of cfDNA from the cfDNA sample. Sequence information can be obtained by parallel sequencing using flow cells. For example, primers for amplification can be covalently attached to slides in the flow cells and then the flow cells can be exposed to reagents for nucleic acids extension and sequencing.

Following the production of an adapter tagged amplicon library, the amplicons are sequenced using high throughput, massively parallel sequencing (i.e., next generation sequencing). In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX IM sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos Biosciences Corp's (Cambridge, MA) single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA, HiSeq 2500, HiSeq 1500, HiSeq 2000, or HiSeq 1000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

High-throughput sequencing of RNA or DNA can also take place using AnyDot-chips (Genovoxx, Germany), which allows monitoring of biological processes (e.g., miRNA expression or allele variability (SNP detection)). For example, the AnyDot-chips allow for 10×-50×enhancement of nucleotide fluorescence signal detection. Other high-throughput sequencing systems include those disclosed in Venter, J., et al., Science 16 Feb. 2001; Adams, M. et al., Science 24 Mar. 2000; and M. J, Levene, et al., Science 299:682-686, January 2003; as well as U.S. Application Pub. No. 2003/0044781 and 2006/0078937.

### Compositions and Methods Including the Modified Magnetic Beads of the Present Technology

Magnetic beads are modified to include at least two different surface modification ligands covalently bonded to the magnetic beads' surfaces. Different surface modification ligands may perform a different type of function.

One of the surface modification ligands is a surface charge ligand that provides a pH-dependent charge state at the surface of the magnetic beads. This surface charge state is modulated by changing the pH of the solution in which the magnetic beads are suspended. When the solution has a pH value less than the pKa of the surface charge ligand, the net charge state at the surface of the magnetic bead is positively charged. When the solution has a pH value greater than the pKa of the surface charge ligand, the surface charge ligand is non-protonated, and the net charge state at the surface of the magnetic bead is essentially neutral. The positive charge state at the surface of the magnetic particle, present at pH values less than the pKA of the surface charge ligand, may electrostatically attract the polyanionic backbone of DNA and RNA. The neutral charge state at the surface of the magnetic bead, present at pH values greater than the pKA of the surface charge ligand, does not exhibit an electrostatic attraction to the polyanionic backbone of DNA or RNA. As an example, the surface charge ligand may have a pKA such that acidic pH induce a positive charge state on the magnetic beads and a neutral pH may induce a neutral charge state on the magnetic beads.

The surface charge ligand may include a heterocyclyl group. The heterocyclyl group may include a five-membered ring or six-membered ring with 1, 2, 3, or 4 heteroatoms. The heteroatoms may be nitrogen. As an example, the heterocyclic group may include an imidazolyl group, a pyridyl group, a pyrimidinyl group, a pyridazinyl group, or a pyrazinyl group.

The heterocyclyl group may be covalently bonded to the surface of the magnetic beads via an amide linkage. The magnetic beads may be provided with carboxylate moieties present on the surface of the magnetic beads and heterocyclic amine molecules may be reacted with the carboxylate moieties resulting in magnetic beads modified with heterocyclic surface charge ligands conjugated to the surface of the magnetic beads via amide linkages. As an example, the heterocyclic amine molecules may include 2-(2-aminoethyl)pyridine and/or 2-(2-aminoethyl)imidazole.

Another surface modification ligand is a connecting ligand including one or more click chemistry reactive groups intended to covalently bond with a click chemistry reactive group on an adapter molecule, an adapter-tagged nucleic acid molecule, and/or an isolated nucleic acid molecule. The connecting ligand forms a covalent bond with the adapter molecule, adapter-tagged nucleic acid molecule, and/or isolated nucleic acid molecule via the click chemistry reactive groups to form a connecting group between the magnetic bead and the adapter molecule, adapter-tagged nucleic acid molecule, and/or isolated nucleic acid molecule.

The connecting ligands may be covalently bonded to the surface of the magnetic beads via an amide linkage. The connecting ligand molecules may include amine moieties that react with carboxylate moieties present on the surface of the magnetic beads to form the amide linkages between the connecting ligand molecules and the magnetic bead surface.

The connecting ligand molecules also include at least one type of click chemistry reactive group intended to undergo a cycloaddition reaction with another type of click chemistry reactive group on the adapter molecule, adapter-tagged nucleic acid molecule, and/or isolated nucleic acid molecule. The cycloaddition reaction may be a bioorthogonal chemical reaction, including a metal-free click reaction. For example, the connector group may be obtained by a strain-promoted azide-alkyne cycloaddition (SPAAC) or an inverse electron-demand Diels-Alder (iEDDA) reaction. The reactive moiety on the adapter and the reactive moiety on the connecting ligand may be any of a variety of complementary reactive moieties that form the connecting group.

The connecting group may include more than one structural element. In other words, the connecting group may result from multiple cycloaddition reactions. For example, the connecting ligands on the magnetic beads may react with a first click chemistry reactive group on an intermediary ligand, and a second click chemistry reactive group on the intermediary ligand may react with the click chemistry reactive group on the adapter molecule, adapter-tagged nucleic acid molecule, and/or isolated nucleic acid molecule.

A non-limiting list of complementary click chemistry reactive group pairings of the present technology includes tetrazinyl moieties and trans-cyclooctene (TCO) moieties; dibenzocyclooctynyl (DBCO) moieties and azido moieties; alkynyl moieties and azido moieties; and sulfanyl moieties and maleimide moieties.

For example, the adapter click chemistry reactive group may include a tetrazinyl group and the corresponding connecting ligand click chemistry reactive group may include a trans-cyclooctene (TCO) moiety or a cycloalkyne moiety. As another example, the adapter click chemistry reactive group may include a TCO moiety or a cycloalkyne moiety and the corresponding connecting ligand click chemistry reactive group may include a tetrazinyl moiety. In any embodiment herein, the tetrazinyl moiety may include tetrazine derivatives including a methyltetrazine (mTZ) moiety. The resulting connecting group may include a dihydropyridazine (from the reaction with alkene) or a pyridazine (from the reaction with alkyne).

For example, the adapter click chemistry reactive group may include a dibenzocyclooctynyl (DBCO) moiety and the corresponding connecting ligand click chemistry reactive group may include an azido moiety. As another example, the adapter click chemistry reactive group may include an azido moiety and the corresponding connecting ligand click chemistry reactive group may include a DBCO moiety. The resulting connecting group may include a triazole group.

For example, the adapter click chemistry reactive group may include an alkynyl moiety and the corresponding connecting ligand click chemistry reactive group may include an azido moiety. As another example, the adapter click chemistry reactive group may include an azido moiety and the corresponding connecting ligand click chemistry reactive group may include an alkynyl moiety. The resulting connecting group may include a triazole group.

For example, the adapter click chemistry reactive group may include a thiol moiety and the corresponding connecting ligand click chemistry reactive group may include a maleimidyl moiety. As another example, the adapter click chemistry reactive group may include a maleimidyl moiety and the corresponding connecting ligand click chemistry reactive group may include a thiol moiety. The resulting connecting group includes a thioether group.

The connecting group may include a hydrophilic spacer. The hydrophilic spacer may include, for example, one or more units, or combinations thereof, selected from ethylene glycol, a carboxylic acid moiety, a sulfonate moiety, a sulfone moiety, a sulfonyl moiety, a sulfonamide moiety, a phosphate moiety, a phosphinate moiety, or an amino group. The hydrophilic spacer may include 1, 2, 3, 4, 5, 6, 7, 8, 9, 10, 11, 12, 13, 14, 15, 16, 17, 18, 19, 20, 21, or 22 units, consecutively or non-consecutively. When the spacer units are non-consecutive, the spacer units may be distributed between cycloaddition groups. For example, the spacer may include about 8 units of polyethylene glycol (PEG).

The relative concentrations of surface charge ligands to connecting ligands on the surface of the modified bead may be about 50%: 50% to about 95%: 5%. For example, the relative concentrations may be about 60%: 40%, 70%: 30%, 80%: 20%, 90%: 10%, or 95%: 5%.

The method of modifying the magnetic beads may include covalently bonding surface charge ligands to the surface of the magnetic beads and covalently bonding connecting ligands to the surface of the magnetic beads. The order in which the surface charge ligands and the connecting ligands are bonded to the beads may vary. In some embodiments, it may be advantageous to modify the magnetic beads with the surface charge ligands first, and then further modify the magnetic beads with the connecting ligands. Alternatively, it may be advantageous to modify the magnetic beads with the connecting ligands first, and then further modify the magnetic beads with the surface charge ligands. In some embodiments, it may be advantageous to modify the magnetic beads with both the surface charge ligands and the connecting ligands simultaneously.

The magnetic beads are modified via solution-phase reactions with the surface charge molecules and the connecting ligand molecules. These reactions may be allowed to proceed for a period of about 1 hour to about 24 hours (including 1 hour, 2 hours, 5 hours, 10 hours, 12 hours, 15 hours, or 24 hours) at a temperature of about 22° C. to about 60° C. (including 25° C. and 36° C.).

The modified magnetic beads or constituents used to make the modified magnetic beads may be present as part of a kit. For example, a kit may include magnetic beads that are surface functionalized with carboxylate moieties, heterocyclic amine molecules to modify the magnetic beads with surface charge ligands, connecting ligand molecules to modify the magnetic beads with connecting ligands, and/or an aqueous buffered solution in which to suspend the magnetic beads. The heterocyclic amine molecules may be present in the kit as a dry solid, in which case the molecules are dissolved in buffered solution prior to reaction with the magnetic beads, or may be present in the kit as a solution. Similarly, the connecting ligand molecules may be present in the kit as a dry solid, in which case the molecules are dissolved in buffered solution prior to reaction with the magnetic beads, or may be present in the kit as a solution.

### Molecular Archiving Methods of the Present Technology

In one aspect, the present disclosure provides a method for archiving nucleic acid molecules isolated from a biological sample comprising (a) isolating a nucleic acid molecule from a biological sample; (b) ligating an adapter to at least one strand of the isolated nucleic acid molecule to form an adapter-tagged nucleic acid molecule, wherein the adapter comprises a click chemistry ligand; and (c) coupling the adapter-tagged nucleic acid molecule to any and all embodiments of the modified magnetic bead described herein to form an adapter-tagged nucleic acid-bead complex, wherein the click chemistry ligand of the adapter forms a covalent linkage with the first click chemistry reactive group on the surface of the modified magnetic bead. The isolated nucleic acid molecule may be double-stranded DNA, single stranded DNA, double-stranded RNA or single stranded RNA. In some embodiments, the double-stranded DNA is genomic DNA, cell-free DNA, or ctDNA. Additionally or alternatively, the isolated nucleic acid molecule may be obtained from a nucleic acid library. In certain embodiments, the method further comprises generating copies of the isolated nucleic acid molecule (e.g., via PCR or isothermal strand displacement) prior to performing step (b).

In some embodiments, the method further comprises contacting the adapter-tagged nucleic acid-bead complex with a blocking agent. Non-limiting examples of suitable blocking agents include, but are not limited to surfactants (e.g., Triton X-100, Tween® 20), polymers (e.g., polyethylene glycol (PEG), polyvinyl alcohol (PVA), polyvinylpyrrolidone (PVP), Ficoll), bovine serum albumin (BSA), coldwater fish gelatine, tryptone casein peptone, casein, milk, serum, and nucleic acid blocking agents (e.g., salmon sperm DNA, calf thymus DNA, yeast tRNA, homopolymer DNA, herring sperm DNA, total human DNA, COT1 DNA). Additionally or alternatively, in some embodiments, the adapter-tagged nucleic acid-bead complex is contacted with the blocking agent for about 30-90 minutes at a temperature of about 20° C.-65° C. In certain embodiments, the adapter-tagged nucleic acid-bead complex is contacted with the blocking agent for about 30-35 minutes, about 35-40 minutes, about 40-45 minutes, about 45-50 minutes, about 50-55 minutes, about 55-60 minutes, about 60-65 minutes, about 65-70 minutes, about 70-75 minutes, about 75-80 minutes, about 80-85 minutes, or about 85-90 minutes at a temperature of about 20° C.-25° C., about 25° C.-30° C., about 30° C.-35° C., about 35° C.-40° C., about 40° C.-45° C., about 45° C.-50° C., about 55° C.-60° C., or about 60° C.-65° C.

Additionally or alternatively, in some embodiments, the method further comprises directly amplifying the adapter-tagged nucleic acid molecule that is coupled to the modified magnetic bead to obtain amplicons. In other embodiments, the method further comprises generating at least one bead-linked copy strand from the adapter-tagged nucleic acid molecule that is coupled to the modified magnetic bead and amplifying the at least one bead-linked copy strand to obtain amplicons. In any of the preceding embodiments of the methods disclosed herein, amplicons may be generated using one or more of the following PCR conditions: an annealing step of about 1, 2, 3, 4, 5, 6, 7, 8, 9 or 10 minutes per cycle, an extension step of about 1, 2, 3, 4 or 5 minutes per cycle, a final extension step of about 5, 6, 7, 8, 9 or 10 minutes.

In any of the preceding embodiments, the method further comprises (a) sequencing the amplicons; (b) detecting at least one genetic alteration in the amplicons, optionally wherein the at least one genetic alteration is selected from the group consisting of a single nucleotide variant (SNV), a copy number variant (CNV), an insertion, a deletion, a duplication, an inversion, a translocation and a gene fusion; and/or (c) enriching the amplicons with a selector comprising a set of oligonucleotides that selectively hybridize to genomic regions of one or more target genes, optionally wherein the one or more target genes correspond to cancer-related genes.

In certain embodiments, the amplicons are sequenced using high throughput, massively parallel sequencing (i.e., next generation sequencing). Methods for performing high throughput, massively parallel sequencing are known in the art. In some embodiments of the method, the high throughput massive parallel sequencing is performed using 454™ GS FLX™ pyrosequencing, reversible dye-terminator sequencing, SOLID sequencing, Ion semiconductor sequencing, Helioscope single molecule sequencing, sequencing by synthesis, sequencing by ligation, or SMRT™ sequencing. In some embodiments, high throughput massively parallel sequencing may be performed using a read depth approach.

In any of the above embodiments, the method further comprises detecting DNA methylation in the adapter-tagged nucleic acid molecule that is coupled to the modified magnetic bead via sodium bisulfite conversion and sequencing, Differential methylation hybridization (DMH), or affinity capture of methylated DNA.

Additionally or alternatively, in some embodiments of the methods disclosed herein, the adapter further comprises a PCR primer binding site, a sequencing primer binding site, or any combination thereof. Additionally or alternatively, in certain embodiments of the methods disclosed herein, the adapter further comprises a sample-specific barcode sequence, wherein the sample-specific barcode sequence comprises 2-20 nucleotides, and/or a detectable label.

In any and all embodiments of the methods disclosed herein, the biological sample comprises no more than 5 ng of cell-free DNA or at least 6-30 ng of cell-free DNA. Additionally or alternatively, in some embodiments of the methods disclosed herein, the biological sample is whole blood, serum, plasma, synovial fluid, lymphatic fluid, ascites fluid, interstitial fluid or a biopsied tissue sample. In certain embodiments, the biological sample is obtained from a patient. Additionally or alternatively, in some embodiments, the patient is diagnosed with ovarian cancer, breast cancer, colon cancer, lung cancer, prostate cancer, gastric cancer, pancreatic cancer, cervical cancer, liver cancer, bladder cancer, cancer of the urinary tract, thyroid cancer, renal cancer, carcinoma, melanoma, head and neck cancer, or brain cancer.

### Selector Design

Somatic mutations, which are mutations that occur in any of the cells of the body except the germ-line cells, can be characteristic of cancer cells. Most human cancers are relatively heterogeneous for somatic mutations in individual genes. A selector can be used to enrich tumor-derived nucleic acid molecules from total genomic nucleic acids. The design of the selector can dictate which mutations can be detected with high probability for a patient with a given cancer. The selector size can also directly impact the cost and depth of sequence coverage. For example, design and use of selectors are described in part in US 2014/0296081 and Newman et al., Nat Med. 20 (5): 548-54 (2014), incorporated herein by reference in their entirety.

The methods disclosed herein may comprise the use of one or more selectors. A selector may comprise a plurality of oligonucleotides or probes that hybridize with one or more genomic regions. The genomic regions may comprise one or more mutated regions. The genomic regions may comprise one or more mutations associated with one or more cancers.

The plurality of genomic regions may comprise different genomic regions. In some embodiments, the plurality of genomic regions may comprise from a few to up to 7500 different genomic regions.

A genomic region may comprise a protein-coding region, or a portion thereof. A protein-coding region may refer to a region of the genome that encodes a protein, e.g., a gene. A genomic region may comprise two or more genes, protein-coding regions, or portions thereof. A gene may also comprise non-coding sequences, such as an intron, or untranslated region (UTR) or portions thereof. In some embodiments, a genomic region does not comprise an entire gene. A genomic region may comprise a pseudogene, a transposon, or a retrotransposon.

A genomic region may comprise a non-protein-coding region. In some embodiments, a non-protein-coding region may be transcribed into a non-coding RNA (ncRNA). In some embodiments, the non-coding RNA may be a transfer RNA (tRNA), ribosomal RNA (rRNA), regulatory RNA, small nuclear RNA (snRNA), small nucleolar RNA (snoRNA), microRNA, small interfering RNA (siRNAs), Piwi-interacting RNA (piRNA), or long ncRNA.

A genomic region may comprise a recurrently mutated region, e.g., a region of the genome, usually the human genome, in which there is an increased probability of genetic mutation in a cancer of interest, relative to the genome as a whole. A recurrently mutated region may also refer to a region of the genome that comprises one or more mutations that is recurrent in the population. A recurrently mutated region may be characterized by a “Recurrence Index” (RI).

The RI generally refers to the number of individual subjects (e.g., cancer patients) with a mutation that occurs within a given kilobase of genomic sequence (e.g., number of patients with mutations/genomic region length in kb). A genomic region may also be characterized by the number of patients with a mutation per exon. Thresholds for each metric (e.g., RI and patients per exon or genomic region) may be selected to statistically enrich for known or suspected drivers of the cancer of interest. Thresholds can also be selected by arbitrarily choosing the top percentile for each metric.

The number of genomic regions in a selector may vary depending on the nature of the cancer. The inclusion of larger numbers of genomic regions may generally increase the likelihood that a unique somatic mutation will be identified. For example, the entire genome of a tumor sample and a genomic sample could be sequenced, and the resulting sequences could be compared to note any differences with the non-tumor tissue.

The library of recurrently mutated genomic regions, or “selector” can be used across an entire population for a given cancer, and does not need to be optimized for each subject.

The method may further comprise a hybridization reaction, e.g., hybridizing the amplicons derived from the adapter-tagged nucleic acid molecules coupled to the modified magnetic beads of the present technology with a selector comprising a set of oligonucleotides that selectively hybridizes to genomic regions of one or more target nucleic acids. In some embodiments, the hybridization reaction may comprise hybridizing the amplicons derived from the adapter-tagged nucleic acid molecules coupled to the modified magnetic beads to a solid support.

The selector may also comprise a set of oligonucleotides. The set of oligonucleotides may hybridize to less than 100 kb and up to 1.5 Megabases (Mb) of the genome. The set of oligonucleotides may be capable of hybridizing at least 5 and up to 500 or more different genomic regions. The selector may also hybridize to a range of different genomic regions, e.g., between about 10 to about 1000 different genomic regions. The selector may also hybridize to a plurality of genomic regions, e.g., about 50 to about 7500 different genomic regions.

A selector may hybridize to a genomic region comprising a mutation that is not recurrent in the population. For example, a genomic region may comprise one or more mutations that are present in a given subject. In some embodiments, a genomic region that comprises one or more mutations in a subject may be used to produce a personalized selector for the subject.

The selector may hybridize to a plurality of genomic regions comprising one or more mutations selected from a group consisting of SNV, CNV, insertions, deletions, and rearrangements.

A selector may hybridize to a mutation in a genomic region known or predicted to be associated with a cancer. A mutation in a genomic region known to be associated with a cancer may be referred to as a “known somatic mutation.” A known somatic mutation may be a mutation located in one or more genes known to be associated with a cancer and may be a mutation present in one or more oncogenes. For example, known somatic mutations may include one or more mutations located in EGFR, KRAS, or BRAF.

Alternatively, a selector may hybridize to a mutation in a genomic region that has not been reported to be associated with a cancer. A genomic region may comprise a sequence of the human genome of sufficient size to capture one or more recurrent mutations.

The methods of the present technology may be directed at cfDNA, which is generally less than about 200 bp in length, and thus a genomic region may be generally less than about 10 kb. Generally the genomic region for a SNV can be quite short, from about 45 bp to about 500 bp in length, while the genomic region for a fusion or other genomic rearrangement may be longer, from about 1 Kb to about 10 Kb in length. A genomic region in a selector may be less than 10 Kb, for example, 100 bp to 10 Kb. In some embodiments, the total sequence covered by the selector is less than about 1.5 megabase pairs (Mb), e.g., 10 kb to 1.5 Mb.

In certain embodiments, a selector useful in the methods of the present technology comprises variants obtained from whole genome sequencing of tumors. For example, the list of variants can be obtained from exome-sequencing nucleic acids from collections of tumor samples, such as a collection of lung squamous cell carcinoma (SCC) tumors or lung adenocarcinoma tumors or any other collections of one or more types of tumors available for sequencing analysis. The sequences may be filtered to eliminate variants located in repeat-rich genomic regions (such as for example, simple repeats, microsatellites, interrupted repeats and segmental duplications). The sequences may also (or instead) be filtered to eliminate variants located in intervals with low mapping rates or low k-mer uniqueness.

Selectors used in the methods disclosed herein can be designed to cover as many patients and mutations per patient as possible with the least amount of genomic space.

In some embodiments, the present disclosure provides a method of creating a selector, i.e., selecting genomic regions to be analyzed in a patient. The selectors may be designed to prioritize inclusion of genomic regions based on the “recurrence index” (RI) metric defined herein. In some embodiments, genomic regions to be included in the selector are exons or smaller portions of an exon containing known lesions. A genomic region to be included comprises the known lesion and is flanked by one or more base pairs to a minimum tile size of 100 bp.

In certain embodiments, genomic regions are ranked by decreasing RI, and those in the highest ranks of both RI and the number of patients per exon are included in the selector. In some embodiments, the highest rank is higher or equal to the top 10%. In this embodiment, the selector has maximized additional patient coverage with minimal space. In some embodiments, the process of selecting genomic regions is repeated under less stringent conditions, i.e., the percentile rank lower than top 10%, e.g., top 33% may be selected. In this embodiment, the method results in including regions that maximally increase the median number of mutations per patient. In some embodiments, inclusion of further genomic regions into a selector is terminated when a predetermined size is reached. In some embodiments, the predetermined desired size is about 100-200 kb. In other embodiments, inclusion of further genomic regions into a selector is terminated when all genomic regions satisfying the filters described above are exhausted.

In some embodiments, the selector comprising genomic regions containing single nucleotide variations (SNVs) further comprises clinically relevant regions containing other types of mutations, e.g., fusions, seed regions, copy number variations (CNVs) and histology classification regions.

The selector can be designed for a specific cancer, for example, non-small cell lung cancer (NSCLC), breast cancer, endometrial uterine carcinoma, etc. The selector can also be designed for a generic class of cancers, e.g., epithelial cancers (carcinomas), sarcomas, lymphomas, melanomas, gliomas, teratomas, etc. The selector can also be designed for a subgenus of cancers, e.g., adenocarcinoma, squamous cell carcinoma, and the like.

The selector may comprise information pertaining to a plurality of genomic regions comprising one or more mutations present in at least one subject suffering from a cancer. For example, the selector may comprise information pertaining to a plurality of genomic regions comprising up to 20 mutations present in at least one subject suffering from a cancer. In some embodiments, the selector may comprise information pertaining to a plurality of genomic regions comprising up to 200 or more mutations present in at least one subject suffering from a cancer. In some embodiments, the one or more mutations within the plurality of genomic regions may be present in at least 1% and up to 20% or more (e.g., up to 95% or more) subjects from a population of subjects suffering from a cancer.

### Examples

The present technology is further illustrated by the following Examples, which should not be construed as limiting in any way.

### Example 1: TCO-2-(2-Aminoethyl)pyridine (AEP) Bead Synthesis Protocol

The modified magnetic beads of the present technology may be prepared as described herein.

Buffers. 2×EDC Coupling Buffer: 200 mM MES pH 4.8; 1×EDC Coupling Buffer: 100 mM MES pH 4.8; Tris High Wash Buffer: 250 mM Tris pH 8+0.01% Tween-20; Tris Low Wash Buffer: 10 mM Tris pH 8+0.01% Tween-20; TE+0.01% Tween-20.

Azido-PEG4-Amine and 2-AEP Coupling Reactions: Bead stocks (SeraMag SpeedBeads Carboxylic Acid, Hydrophobic (50 mg/ml): 200 μl) were mixed well and 10 mg of beads were transferred to a clean microfuge tube. The beads were magnetically collected and the supernatants were discarded. The beads were washed with 3×500 μl 1×EDC Coupling Buffer. The last wash was discarded and the beads were resuspended in 250 μl 1×EDC Coupling Buffer.

The starting concentration of neat azido-PEG4-amine was 4.167 μmol/μl. 3.5 μl of the azido-PEG4-amine reagent was diluted with 57.8 μl 1×EDC Coupling Buffer to make a working concentration of 0.238 μmol/μl.

1 g (8.185 mmol)₂-(2-aminoethyl)pyridine (2-AEP, FW 122.17) was dissolved in 1×EDC Coupling Buffer to a final concentration of 5.264 μmol/μl. 35 μl 2-AEP was diluted with 739 μl 1×EDC Coupling Buffer to make a solution at 0.238 μmol/μl.

PEG-Azido: AEP reaction mixes were prepared as follows: 20% PEG4-azide: 50 μl Azido-PEG4-Amine+200 μl 2-AEP. Immediately before use, 34.2 mg EDC was dissolved in 1×EDC Coupling Buffer142.3 μl to make a 1.25M solution. The EDC was removed from the freezer and warmed to room temperature before opening.

To the bead aliquot, 40 μl (50 μmol) 1.25M EDC was added and mixed briefly. 210 μl (50 μmol amine) of the Azido-PEG4-Amine/2-AEP mix was added and vortexed to mix. Final bead concentration was 20 mg/ml. In a thermal mixer, the samples were shaken at 1500 rpm while heating to 37° C. The reaction was allowed to proceed overnight.

Conjugation of AEP to carboxylate-modified magnetic particles produces a magnetic substrate where the surface charge can be modulated by buffer pH. Buffer pH values lower than the pKa of the AEP results in a positively charged surface, whereas pH values above the pKa results in a non-protonated and therefore neutral surface. See FIG. 1. At lower pH values, the positive surface of the magnetic particle can electrostatically attract the polyanionic backbone of DNA and RNA.

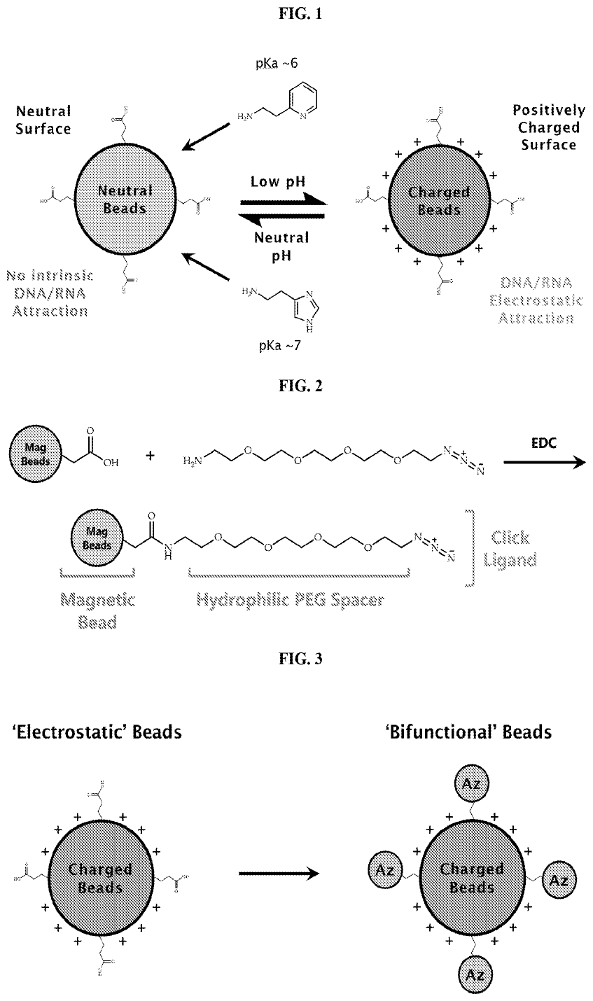

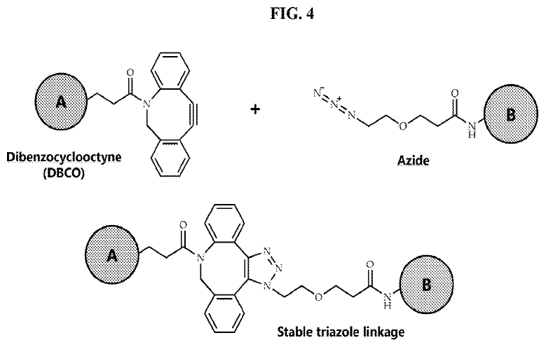

In addition to the AEP conjugated to the bead surface, click chemistry ligands (e.g., azide groups) are added to the bead surface using standard conjugation chemistry to create a bifunctional reagent. See FIGS. 2-4, 7.

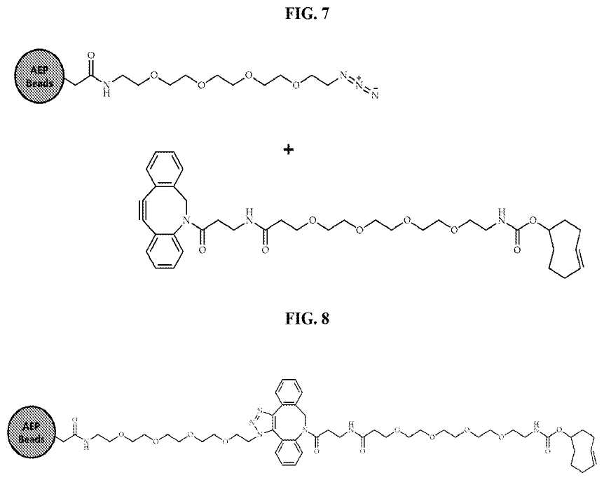

Bead Washing & TCO Coupling. After 18 hours of reaction time, the beads were washed. The beads were magnetically collected and the supernatants were discarded. The beads were resuspended in 0.5 ml Tris High Wash Buffer and the wash was discarded. 0.5 ml Tris High Wash Buffer was added and the mixture was nutated at room temperature for 30 min. The beads were magnetically collected and supernatant was discarded. The beads were washed with 3×500 μl Tris Low Wash Buffer. The beads were resuspended in 250 μl water and 250 μl DMSO (50% final) was added. The beads were mixed and collected, and the wash was discarded. The beads were washed 2× with 500 μl DMSO and resuspended in 487.5 μl DMSO. 12.5 μl TCO-PEG4-DBCO (185 mM in DMSO) was added to the beads and mixed (FIG. 7). Final reagent (FIG. 8) concentration was 4.63 mM. The beads were mutated at room temperature overnight.

Bead Washing and Storage. After 24 hours, the beads were collected and the supernatants were discarded. The beads were washed 3× with 500 μl DMSO and resuspended in 250 μl DMSO, and 250 μl TE/Tween was added. The beads were mixed and collected and the supernatants were discarded. The beads were washed 3× with 500 μl TE+0.01% Tween-20. After the last wash, the beads were stored in TE+0.01% Tween-20 at a concentration of 10 mg/ml (1 ml final).

### Example 2: Synthesis of methyltetrazine (mTz) Labelled Adapters

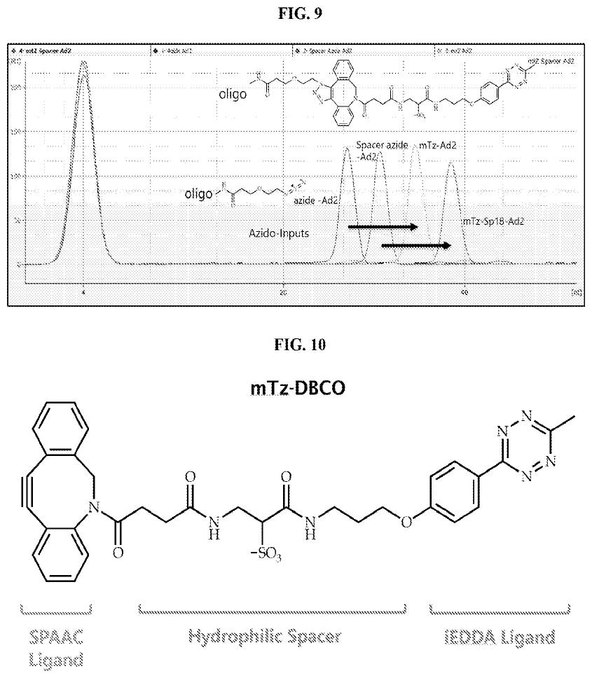

Commercially available DNA oligonucleotides may be modified so as to be compatible with the modified magnetic beads of the present technology. In one example, a mTz moiety may be included onto an azide labelled nucleic acid adapter. See FIG. 9.

Reagents. 20 mM mTz-DBCO; 100 μM Azide Prism Adapter2, 5M NaCl, 100% ethanol, Small RNA Bioanalyzer kit

Procedure. 10 μL of 100 μM Azide Prism Adapter 2 was aliquoted and 1 μL of 20 mM mTz-DBCO was added. The mixture was vortexed, spun down and incubated at room temperature overnight on a rotator. 1.1 μL of 5M NaCl was added to the reaction (1/10th of reaction volume). 36.3 μL of 100% ethanol was added to the reaction (3× reaction volume). The reaction was mixed and incubated at −20° C. for 30 minutes. The mixture was spun at full speed in a pre-chilled 4° C. microcentrifuge for 10 minutes. The supernatant was carefully removed without disturbing the pellet. 100 μL of freshly prepared 80% ethanol was added to the tube and the tube was gently inverted and spun at full speed in a pre-chilled 4° C. microcentrifuge for 5 minutes. The pellet was subjected to an additional ethanol wash for a total of 2 washes. The pellet was spun down and the supernatant was carefully removed without disturbing the pellet. The sample was air dried for 5 minutes and dissolved in 10 μL of water. 1 μL of 100 μM Azide Prism Adapter (control) and 1 μL of mTz Prism Adapter 2 were run on Nanodrop. The A260s was recorded and the concentration of mTz Prism Adapter 2 was determined. 1 μL of Azide Prism Adapter 2 and 1 μL of mTz Prism Adapter 2 were diluted down to 250 fmol/μL and the dilutions were run on small RNA Bioanalyzer to ensure a full shift occurred. See FIG. 9.

### Example 3: Bead Link (BLINK) Method for Molecular Archiving of DNA

This Example provides the protocol for capturing nucleic acids (e.g., DNA, RNA) onto the surface of the modified magnetic beads of the present technology and generate nucleic acid libraries attached to the modified magnetic beads for iterative sampling and long-term storage.

Reagents. mTz-Azide Prism Adapter 2 prepared library after second ligation cleanup; 20% TCO AEP Beads; 0.5 M Sodium Acetate/Tween (0.5M sodium acetate, 0.1% Tween), pH 4.0; 0.05 M NaOH; 0.1 M Tris-HCl, pH 7.0; TE/Tween (10 mM Tris, 0.1 mM EDTA, 0.01% Tween); Dynamag-2 Magnet (Thermo Fisher #12321D); 1.5 mL Eppendorf tubes.

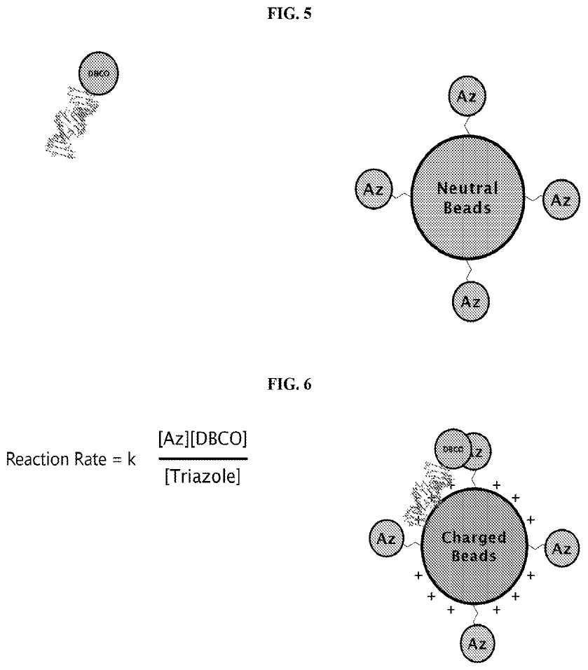

Procedure. 5 μL of TCO AEP beads per sample were aliquoted into individual 1.5 mL Eppendorf tubes, spun down in a microcentrifuge and placed on a Dynamag-2 Magnet. The supernatant was discarded. 18 μL of mTz-Azide Prism Adapter 2 prepared library was added to the TCO AEP beads, vortexed and spun down. The mixture was incubated for 1 minute at room temperature. 2 μL of 0.5 M Sodium Acetate/Tween was added to the bead mixture, vortexed and spun down. The mixture was incubated for 2 minutes at room temperature. The slight drop in pH charges the surface of the beads, creating an attractive force between the TCO AEP bead and the mTz labeled DNA molecules. Once the DNA was electrostatically adhered to the TCO AEP bead, the click chemistry mTz group on the DNA molecule reacted with the corresponding TCO reactive group on the magnetic bead. See FIG. 6. Specifically, the electrostatic attraction ‘concentrates’ the click substrates, resulting in a dramatic increase in the reaction rate of the reagents. The overall process creates an incredibly fast conjugation reaction. Under neutral conditions however, there is no attractive force between the labeled DNA and the magnetic particle. FIG. 5.

The mixture was placed on a magnet until the supernatant was clear, and the supernatant was discarded. 5 μL of 0.05 M NaOH was added to the TCO AEP beads, resuspended, vortexed and spun down. The mixture was incubated for 1 minute at room temperature. The mixture was placed on a magnet until the supernatant was clear, and the supernatant was discarded. The NaOH wash step was repeated twice for a total of three washes.

5 μL of 0.1M Tris-HCl, pH 7.0 was added to the TCO AEP beads. The beads were resuspended, vortexed and spun down. The mixture was placed on a magnet until the supernatant was clear, and the supernatant was discarded. 50 μL of TE/Tween was added to the beads. The beads were resuspended, vortexed and spun down. The mixture was placed on a magnet until the supernatant was clear, and the supernatant was discarded. The TE/Tween wash steps were repeated twice for a total of three washes. The TCO AEP beads ligated to the nucleic acids (e.g., DNA, RNA) of the prepared library were spun down and the supernatant was discarded. The TCO AEP beads were resuspended in 20 μL H₂O.

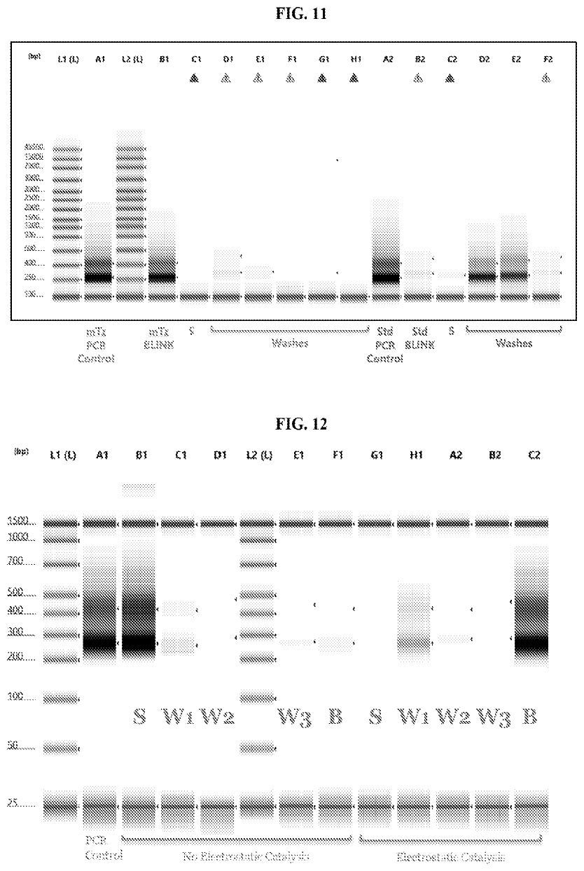

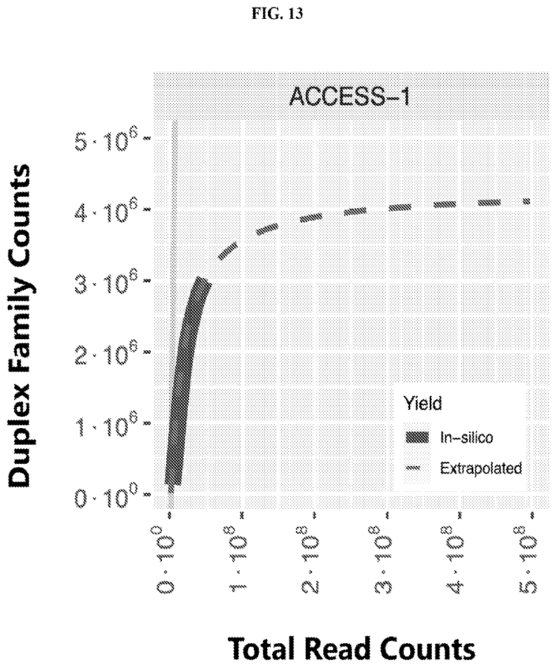

Results. An amplicon was made with a methyltetrazine (mTz) click group on one strand. An un-labeled amplicon that does not have the click group was used as a control (Standard or Std). The mTz amplicon and Std amplicon underwent electrostatic catalysis. These amplicons were then washed five times with TE/SDS/NaCl. The supernatant(S) and washes were saved and PCR was performed to determine the amount of material on the modified magnetic beads (mTz BLINK and Std BLINK), and the amount of material left in the supernatant and washes. PCR controls were run to make sure the reagents were not faulty. As shown in FIG. 11, the modified magnetic beads successfully captured most of the nucleic acid material including the click group (mTz BLINK) that underwent electrostatic catalysis, whereas most of the unlabeled nucleic acid material was lost in the wash fractions.

An amplicon was made with a methyltetrazine (mTz) click group on the both strands. One set did not have the binding buffer added to it (No Electrostatic Catalysis), whereas the other did (Electrostatic Catalysis). The reactions were allowed to react overnight. These amplicons were then washed three times with 0.5 M NaOH/Tween. The supernatant(S) and washes (W1, W2, and W3) were saved and PCR was performed to see the amount of material on the bead (B), and the amount of material left in the supernatant and washes. A PCR control was run to make sure the reagents were not faulty. As shown in FIG. 12, DNA did not bind to the beads in the absence of electrostatic catalysis and most of the mTz labelled nucleic acid material was present in the supernatant(S). In contrast, most of the mTz labelled nucleic acid material was bound to the modified magnetic beads that were subjected to electrostatic catalysis. Accordingly, in order for the DNA to be efficiently captured onto the modified magnetic beads of the present technology, electrostatic catalysis is required.

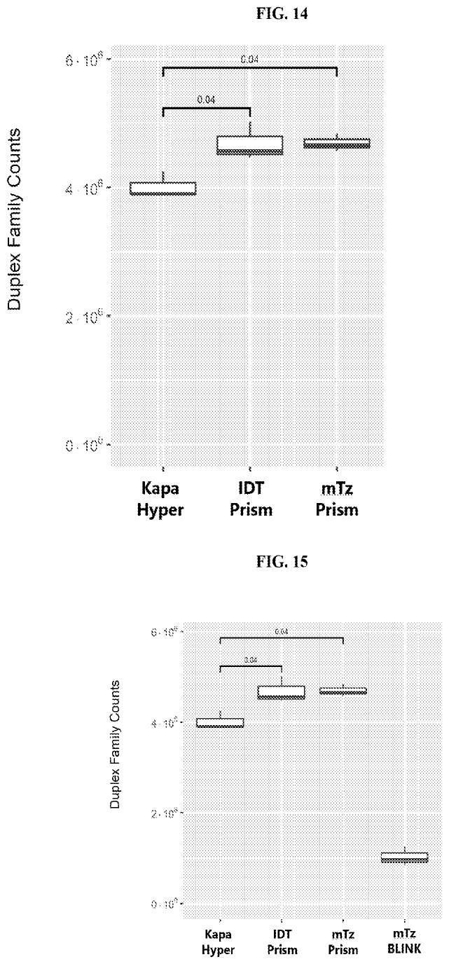

In order to ensure that the click chemistry groups on the modified magnetic beads did not interfere with library preparation, NGS libraries were made using the Kapa Hyper Kit, the standard IDT Prism kit, and the IDT prism kit with a mTz modified adapter, and sequenced. As shown in FIG. 14, the click chemistry ligands on the modified magnetic beads do not affect the performance of the library preparation kit as can be seen by the similar duplex family count as the standard IDT Prism kit. As such, inclusion of the click chemistry group does not negatively affect library prep. The extrapolated library complexity is at a total read depth of 5×10⁸

These results demonstrate that the methods of present technology are useful in conjugating labelled DNA molecules to modified magnetic beads via electrostatic catalysis with a high degree of efficiency and without interfering with nucleic acid library preparation.

### Example 4: Attachment of DNA Libraries to Beads for Iterative Sampling and Long-term Storage (BLINK plus Direct Amplification from Beads)

Reagents. mTz-Azide Prism Adapter 2 prepared library after second ligation cleanup; 20% TCO AEP Beads; 0.5 M Sodium Acetate/Tween, pH 4.0 (0.5M sodium acetate, 0.1% Tween), pH 4.0; 0.05 M NaOH; 0.1 M Tris-HCl, pH 7.0; TE/Tween (10 mM Tris, 0.1 mM EDTA, 0.01% Tween); Dynamag-2 Magnet (Thermo Fisher #12321D); 1.5 mL Eppendorf tubes

Procedure. 5 μL of TCO AEP beads per sample were aliquoted into individual 1.5 mL Eppendorf tubes, spun down in a microcentrifuge and placed on a Dynamag-2 Magnet. The supernatant was discarded. 18 μL of mTz-Azide Prism Adapter 2 prepared library was added to the TCO AEP beads, vortexed and spun down. The mixture was incubated for 1 minute at room temperature. 2 μL of 0.5 M Sodium Acetate/Tween was added to the bead mixture, vortexed and spun down. The mixture was incubated for 2 minutes at room temperature.

The mixture was placed on a magnet until the supernatant was clear, and the supernatant was discarded. 5 μL of 0.05 M NaOH was added to the TCO AEP beads, resuspended, vortexed and spun down. The mixture was incubated for 1 minute at room temperature. The mixture was placed on a magnet until the supernatant was clear, and the supernatant was discarded. The NaOH wash step was repeated twice for a total of three washes.

5 μL of 0.1M Tris-HCl, pH 7.0 was added to the TCO AEP beads. The beads were resuspended, vortexed and spun down. The mixture was placed on a magnet until the supernatant was clear, and the supernatant was discarded. 50 μL of TE/Tween was added to the beads. The beads were resuspended, vortexed and spun down. The mixture was placed on a magnet until the supernatant was clear, and the supernatant was discarded. The TE/Tween wash steps were repeated twice for a total of three washes. The TCO AEP beads ligated to the nucleic acids (e.g., DNA, RNA) of the prepared library were spun down and the supernatant was discarded. The TCO AEP beads were resuspended in 20 μL H₂O.

5 μL of amplification primer from UDI primers and 25 μL of 2×Kapa Hifi Polymerase Mix were added to the 20 μL solution containing the TCO AEP beads ligated to the nucleic acid library. Direct amplification was performed on the beads using the following conditions:

| | Temperature | | | |
|---|---|---|---|---|
| Step | (° C.) | Duration | | Cycles |
| Initial denaturation | 98 | 3 | minutes | 1 |
| Denaturation | 98 | 1 | minute | 10 |
| Annealing | 60 | 2 | minutes | |
| Extension | 72 | 1 | minute | |
| Final Extension | 72 | 5 | minutes | 1 |

| | | | |
|---|---|---|---|
| Hold | 4 | Hold | 1 |

Results. NGS libraries were made using the Kapa Hyper Kit, the standard IDT Prism kit, and the IDT prism kit with a mTz modified adapter. A portion of the NGS library that was generated with the IDT prism kit with a mTz modified adapter was ligated onto the surface of the modified magnetic beads of the present technology (mTz BLINK). In these experiments, the library was amplified directly from the modified magnetic beads of the present technology. The data recovered from the nucleic acid library archived on the modified magnetic beads (mTz BLINK) is lower in complexity relative to the libraries made using standard library preparation kits. See FIG. 15. Since archiving the DNA onto the bead surface appears efficient (see FIGS. 11-12), the lower library complexity is most likely due to the sequencing inefficiency when the archived nucleic acid molecules are directly amplified off the modified magnetic beads.

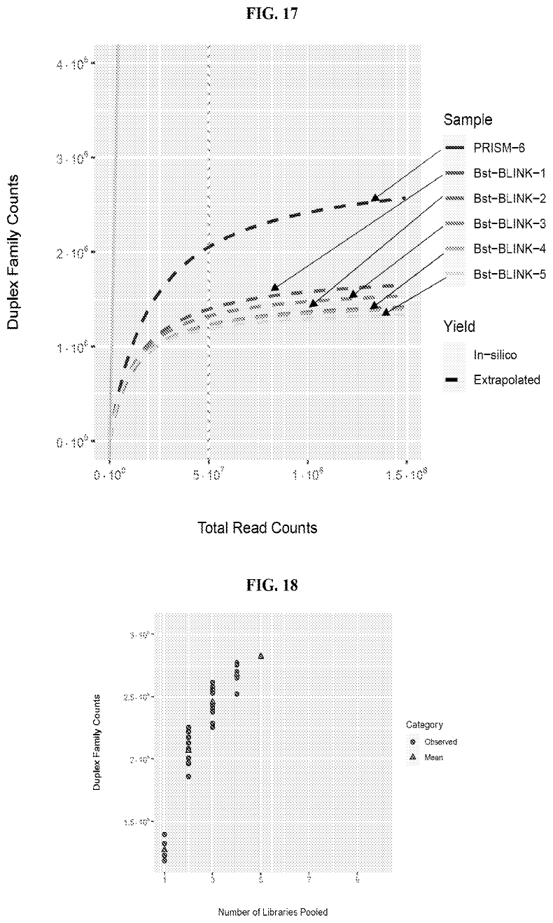

Five individual sequencing libraries were iteratively generated from the same modified magnetic bead. None of the libraries by themselves show complexity (duplex family content) that equals the control ‘Prism’ preparation (NGS library made with standard IDT prism kit). See FIG. 17. Information content in each library is additive (to a point). However, combining these libraries together shows that each library contains complementary DNA information and produces better duplex yield. See FIG. 18. Therefore multiple sampling will improve the complexity of the final library and may bring the value close to that of a control NGS library made with standard IDT prism kit.

### Example 5: Attachment of DNA Libraries to Beads for Iterative Sampling and Long-term Storage (BLINK plus Creating a Bead-linked Copy Strand and Amplifying Bead-linked Copy Strand)

Reagents. mTz-Azide Prism Adapter 2 prepared library after second ligation cleanup (Eluted in 20.5 μL TE); 20% TCO AEP Beads; 0.5 M Sodium Acetate/Tween, pH 4.0 (0.5M sodium acetate, 0.1% Tween), pH 4.0; 0.05 M NaOH/Tween (0.05M NaOH, 0.1% Tween); 0.3 M Tris-HCl, pH 7.0; TE/Tween (10 mM Tris, 0.1 mM EDTA, 0.01% Tween); Dynamag-2 Magnet (Thermo Fisher #12321D); 1.5 mL Eppendorf tubes; 10×Isothermal Reaction Buffer (New England Biosciences #M0537S); 40 μM Prism Block V2; 10 mM dNTPs; Bst 2.0 (New England Biosciences #M0537S); 2×Kapa Hifi Mix (Roche #07958935001); xGen UDI Primers (Integrated DNA Technologies #10005921).

Procedure. The Prism prepped library after ligation 2 cleanup was placed on a magnet and the supernatant was transferred to a new Eppendorf tube. Then, 2.5 μL 10×Isothermal Reaction Buffer, 0.5 μL of 40 μM Prism Block V2, 0.5 μL of 10 mM dNTPs, and 1 μL of Bst 2.0 were added to the Eppendorf tube. The sample was incubated at 55° C. for one hour on the thermomixer.

5 μL of 20% TCO AEP beads were aliquoted (per sample) into individual 1.5 mL Eppendorf tubes and spun down in a microcentrifuge. The mixture was placed on a Dynamag-2 Magnet and the supernatant was discarded. The entire volume of Bst-treated sample was added onto beads. 2 μL of H₂O and 3 μL of 0.5 M Sodium Acetate/Tween were added to the bead mixture. The bead mixture were vortexed, spun down and incubated for 2 minutes at room temperature. The bead mixture was placed on a magnet until supernatant is clear, and supernatant was discarded.

5 μL of 0.05 M NaOH was added to the beads. The beads were resuspended, vortexed, spun down and incubated for 1 minute at room temperature. The sample was placed on a magnet until supernatant was clear, and the supernatant was saved. The NaOH wash steps were repeated twice for a total of three washes and all saved supernatants were combined.

5 μL of 0.3M Tris-HCl, pH 7.0 was added to the beads. The beads were resuspended, vortexed, and spun down. The sample was placed on a magnet until supernatant was clear, and the supernatant was combined with previously saved combined washes.

50 μL of TE/Tween was added to the beads. The beads were resuspended, vortexed, and spun down. The sample was placed on a magnet until supernatant was clear, and the supernatant was discarded. The beads were resuspended in 20 μL of H₂O.

5 μL of amplification primer from UDI primers and 25 μL of 2×Kapa Hifi Polymerase Mix were added to a mixture comprising the saved combined washes which contain the bead-linked copy strands and bead samples. The mixture was amplified using the following conditions:

| | Temperature | | | |
|---|---|---|---|---|
| Step | (° C.) | Duration | | Cycles |
| Initial denaturation | 98 | 45 | seconds | 1 |
| Denaturation | 98 | 15 | seconds | 10 |
| Annealing | 60 | 30 | seconds | |
| Extension | 72 | 30 | seconds | |
| Final Extension | 72 | 1 | minute | 1 |

| | | | |
|---|---|---|---|
| Hold | 4 | Hold | 1 |

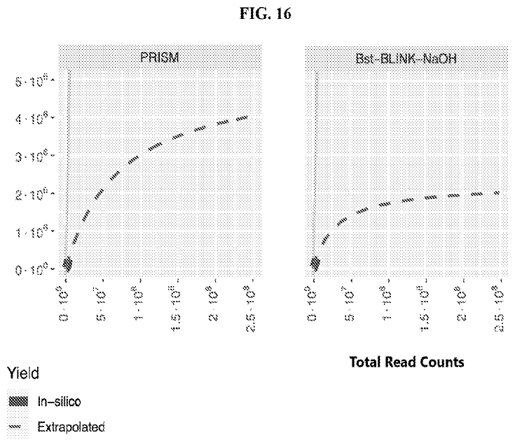

Results. During the modified library preparation, Bst 2.0 was used to separate the strands of the original DNA molecules captured on the beads and a primer was added to create bead-linked copy strands. NaOH washes were then performed to strip the bead-linked copy strand before PCR. As shown in FIG. 16, generation of a bead-linked copy strand improved reading of the archived DNA material from the modified magnetic beads.

### Example 6: Attachment of DNA Libraries to Beads for Iterative Sampling and Long-term Storage with Bst Strand Separation with Pre-Amplification Strand Separation

Reagents. mTz-Azide Prism Adapter 2 prepared library after second ligation cleanup (Eluted in 10 μL TE); 20% TCO AEP Beads; 0.5 M Sodium Acetate/Tween, pH 4.0 (0.5M sodium acetate, 0.1% Tween), pH 4.0; 0.05 M NaOH/Tween (0.05M NaOH, 0.1% Tween); 0.3 M Tris-HCl, pH 7.0; TE/Tween (10 mM Tris, 0.1 mM EDTA, 0.01% Tween); Dynamag-2 Magnet (Thermo Fisher #12321D); 1.5 μL Eppendorf tubes; PCR strip tubes and caps; 10 μM BLINK PreAmp1 (+) Primer; 10 μM BLINK PreAmp1 (−) Primer; 2×Kapa Hifi Mix (Roche #07958935001); xGen UDI Primers (Integrated DNA Technologies #10005921); Ampure Beads; 80% Ethanol

Procedure. The Prism prepped library after ligation 2 cleanup was placed on a magnet and the supernatant was transferred to a new PCR tube. 1.25 μL of 10 μM BLINK PreAmp1 (+) Primer, 1.25 μL of 10 μM BLINK PreAmp1 (−) Primer, 12.5 μL of 2×Kapa Hifi Mix were added to the PCR tube and PCR was performed under the following conditions:

| | Temperature | | | |
|---|---|---|---|---|
| Step | (° C.) | Duration | | Cycles |
| Initial denaturation | 98 | 45 | seconds | 1 |
| Denaturation | 98 | 15 | seconds | 4 |
| Annealing | 60 | 30 | seconds | |
| Extension | 72 | 30 | seconds | |
| Final Extension | 72 | 1 | minute | 1 |

| | | | |
|---|---|---|---|
| Hold | 4 | Hold | 1 |

5 μL of 20% TCO AEP beads were aliquoted (per sample) into individual 1.5 mL Eppendorf tubes and spun down in a microcentrifuge. The mixture was placed on a Dynamag-2 Magnet and the supernatant was discarded. The entire volume of pre-amplified sample was added onto beads. 15 μL of H₂O and 10 μL of 0.5 M Sodium Acetate/Tween were added to the bead mixture. The bead mixture were vortexed, spun down and incubated for 2 minutes at room temperature. The bead mixture was placed on a magnet until supernatant is clear, and supernatant was discarded.

5 μL of 0.05 M NaOH was added to the beads. The beads were resuspended, vortexed, spun down and incubated for 1 minute at room temperature. The sample was placed on a magnet until supernatant was clear, and the supernatant was saved. The NaOH wash steps were repeated twice for a total of three washes and all saved supernatants were combined.

5 μL of 0.3M Tris-HCl, pH 7.0 was added to the beads. The beads were resuspended, vortexed, and spun down. The sample was placed on a magnet until supernatant was clear, and the supernatant was combined with previously saved combined washes (Washes sample). The Washes sample was placed on ice.

50 μL of TE/Tween was added to the beads. The beads were resuspended, vortexed, and spun down. The sample was placed on a magnet until supernatant was clear, and the supernatant was discarded. The beads were resuspended in 20 μL of H₂O. 2.5 μL of 10 μM BLINK PreAmp (+) Primer, 2.5 μL of 10 μM BLINK PreAmp (−) Primer, and 25 μL of 2×Kapa Hifi Mix were added to the bead sample and PCR was performed with the following parameters:

| | Temperature | | | |
|---|---|---|---|---|
| Step | (° C.) | Duration | | Cycles |
| Initial denaturation | 98 | 45 | seconds | 1 |
| Denaturation | 98 | 15 | seconds | 6 |
| Annealing | 60 | 30 | seconds | |
| Extension | 72 | 30 | seconds | |
| Final Extension | 72 | 1 | minute | 1 |

| | | | |
|---|---|---|---|
| Hold | 4 | Hold | 1 |

The sample was placed on a magnet and the supernatant was transferred to a new PCR tube.

The beads were resuspended in 50 μL TE/Tween and store in 4° C. 50 μL of Ampure beads were transferred to supernatant, mixed well and incubated at room temperature for 10 minutes. The mixture was placed on a magnet and supernatant was discarded. 160 μL of 80% ethanol was added to the mixture and then incubated for 30 seconds. The mixture was placed on a magnet and supernatant was discarded. The bead mixture was eluted in 20 μL H₂O.

Washes sample and beads sample were combined 5 μL of amplification primer from UDI primers and 25 μL of 2×Kapa Hifi Polymerase Mix were added to the combined sample, and PCR was performed using the following conditions:

| | Temperature | | | |
|---|---|---|---|---|
| Step | (° C.) | Duration | | Cycles |
| Initial denaturation | 98 | 45 | seconds | 1 |
| Denaturation | 98 | 15 | seconds | 4 |
| Annealing | 60 | 30 | seconds | |
| Extension | 72 | 30 | seconds | |
| Final Extension | 72 | 1 | minute | 1 |

| | | | |
|---|---|---|---|
| Hold | 4 | Hold | 1 |

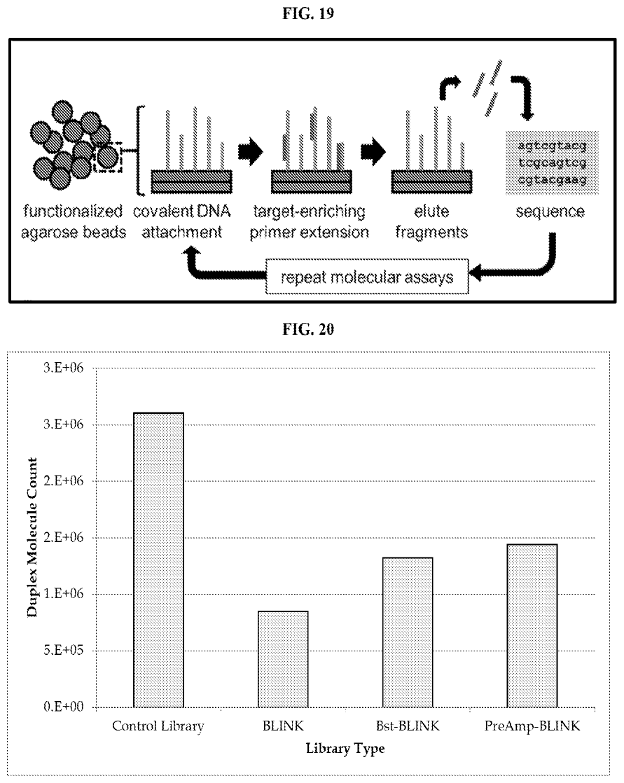

As shown in FIG. 20, strand separation prior to linking adapter-tagged nucleic acids to the modified magnetic beads of the present technology improves duplex count. Compared to a control library, a bead linked (BLINK)-archived nucleic acid library shows less duplex molecule content (first and second columns). When the original DNA strands are separated by either isothermal strand displacement/polymerization (for example, using the Bst2.0 enzyme) or by limited rounds of PCR (e.g. pre-amplification), the resulting archived libraries show higher duplex content (columns 3 and 4).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method comprising
(a) conjugating a heterocyclic amine to a surface of a magnetic bead to obtain a modified magnetic bead having a pH dependent charge state; and
(b) conjugating a reagent comprising a first click chemistry reactive group to the surface of the modified magnetic bead,
wherein the modified magnetic bead is configured to attach to a nucleic acid molecule comprising a second click chemistry reactive group, wherein the second click chemistry reactive group of the nucleic acid molecule forms a covalent linkage with the first click chemistry reactive group on the surface of the modified magnetic bead, optionally wherein step (a) and step (b) occur simultaneously or sequentially.

2. (canceled)

3. The method of claim 1, wherein the pH dependent charge state of the modified magnetic bead is positive at an acidic pH and neutral at a neutral pH.

4. The method of claim 1, wherein the surface of the modified magnetic bead comprises at least one carboxylate-moiety, optionally wherein the reagent comprising the first click chemistry reactive group is conjugated to the surface of the modified magnetic bead via the at least one carboxylate-moiety.

5. (canceled)

6. The method of claim 1, wherein the reagent further comprises a hydrophilic spacer, optionally wherein the hydrophilic spacer comprises one or more of an ethylene glycol moiety (e.g., PEG), a sulfonate moiety, a sulfone moiety, a sulfonyl moiety, a sulfonamide moiety, a phosphate moiety, or a phosphinate moiety.

7. The method of claim 1, wherein the reagent further comprises one or more functional moieties selected from among dibenzocyclooctyne (DBCO), trans-cyclooctene (TCO), triazole, methyltetrazine, thiol or maleimide.

8. The method of claim 1, wherein:
the first click chemistry reactive group is methyltetrazine and the second click chemistry reactive group is trans-cyclooctene (TCO);
the first click chemistry reactive group is azide and the second click chemistry reactive group is dibenzocyclooctyne (DBCO);
the first click chemistry reactive group is azide and the second click chemistry reactive group is alkyne; or
the first click chemistry reactive group is maleimide and second click chemistry reactive group is thiol.

9. The method of claim 1, wherein:
the first click chemistry reactive group is trans-cyclooctene (TCO) and the second click chemistry reactive group is methyltetrazine;
the first click chemistry reactive group is dibenzocyclooctyne (DBCO) and the second click chemistry reactive group is azide;
the first click chemistry reactive group is alkyne and the second click chemistry reactive group is azide; or
the first click chemistry reactive group is thiol and second click chemistry reactive group is maleimide.

10. The method of claim 1, wherein the heterocyclic amine is 2-(2-aminoethyl)pyridine or 2-(2-aminoethyl)imidazole.

11. A modified magnetic bead produced by the method of claim 1.

12. A method for archiving nucleic acid molecules isolated from a biological sample comprising
(a) isolating a nucleic acid molecule from a biological sample;
(b) ligating an adapter to at least one strand of the isolated nucleic acid molecule to form an adapter-tagged nucleic acid molecule, wherein the adapter comprises a click chemistry ligand; and
(c) coupling the adapter-tagged nucleic acid molecule to the modified magnetic bead of claim 11 to form an adapter-tagged nucleic acid-bead complex, wherein the click chemistry ligand of the adapter forms a covalent linkage with the first click chemistry reactive group on the surface of the modified magnetic bead.

13. The method of claim 12, further comprising contacting the adapter-tagged nucleic acid-bead complex with a blocking agent.

14. The method of claim 12, further comprising directly amplifying the adapter-tagged nucleic acid molecule that is coupled to the modified magnetic bead to obtain amplicons.

15. The method of claim 12, further comprising generating at least one bead-linked copy strand from the adapter-tagged nucleic acid molecule that is coupled to the modified magnetic bead and amplifying the at least one bead-linked copy strand to obtain amplicons.

16. The method of claim 12, wherein the isolated nucleic acid molecule is double-stranded DNA, single stranded DNA, double-stranded RNA or single stranded RNA, optionally wherein the double-stranded DNA is genomic DNA, cell-free DNA, or ctDNA.

17. (canceled)

18. The method of claim 14, further comprising
(a) sequencing the amplicons;
(b) detecting at least one genetic alteration in the amplicons, optionally wherein the at least one genetic alteration is selected from the group consisting of a single nucleotide variant (SNV), a copy number variant (CNV), an insertion, a deletion, a duplication, an inversion, a translocation and a gene fusion; and/or
(c) enriching the amplicons with a selector comprising a set of oligonucleotides that selectively hybridize to genomic regions of one or more target genes, optionally wherein the one or more target genes correspond to cancer-related genes.

19. The method of claim 12, further comprising detecting DNA methylation in the adapter-tagged nucleic acid molecule that is coupled to the modified magnetic bead via sodium bisulfite conversion and sequencing, Differential methylation hybridization (DMH), or affinity capture of methylated DNA.

20. The method of claim 12, wherein the adapter further comprises a PCR primer binding site, a sequencing primer binding site, or any combination thereof.

21. The method of claim 12, wherein the adapter further comprises a sample-specific barcode sequence, wherein the sample-specific barcode sequence comprises 2-20 nucleotides or wherein the adapter further comprises a detectable label.

22. (canceled)

23. The method of claim 12, wherein the biological sample
comprises no more than 5 ng of cell-free DNA or at least 6-30 ng of cell-free DNA; or
is whole blood, serum, plasma, synovial fluid, lymphatic fluid, ascites fluid, interstitial fluid or a biopsied tissue sample; or
is obtained from a patient.

24. (canceled)

25. (canceled)

26. The method of claim 12, wherein the patient is diagnosed with ovarian cancer, breast cancer, colon cancer, lung cancer, prostate cancer, gastric cancer, pancreatic cancer, cervical cancer, liver cancer, bladder cancer, cancer of the urinary tract, thyroid cancer, renal cancer, carcinoma, melanoma, head and neck cancer, or brain cancer.
